(19) United States
(12) Patent Application Publication
(10) Pub. No.: US 2009/0181989 A1
(43) Pub. Date: Jul. 16, 2009

(54) Purine Compunds as HSP90 Protein Inhibitors for the Treatment of Cancer

(75) Inventors: Paul Andrew BROUGH, Berkshire (GB); Martin DRYSDALE, Berkshire (GB); Xavier BARRIL-ALONSO, Barcelona (ES)

Correspondence Address: BANNER & WITCOFF, LTD., 1100 13th STREET, N.W., SUITE 1200, WASHINGTON, DC, 20005-4051, US

(73) Assignees: VERNALIS ( R & D) LTD., Winnersh (GB); CANCER RESEARCH TECHNOLOGY LTD, London (GB); THE INSTITUTE OF CANCER, London (GB)

(21) Appl. No.: 12/394,891

(22) Filed: Feb. 27, 2009

Related U.S. Application Data
(63) Continuation of application No. 12/067,232.

(30) Foreign Application Priority Data
Sep. 20, 2005 (GB) 0519245.5

(51) Int. Cl. A61K 31/52; C07D 473/40; C07D 473/36; C07D 473/28; A61K 31/522; A61P 31/12; A61P 29/00; A61P 31/10; A61P 3/10; A61P 35/00

(52) U.S. Cl. 514/263.3; 544/264; 544/265; 514/263.1

(57) ABSTRACT

Compounds of formula (I) are inhibitors of HSP90, and of utility in the treatment of, for example, cancers:

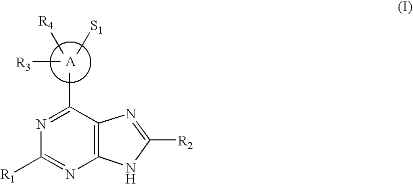

wherein ring A is an aryl or heteroaryl ring or ring system; R₁ is hydrogen, fluoro, chloro, bromo, or a radical of formula (IA): —X-Alk¹-(Z)ₘ-(Alk²)ₙ-Q (IA) wherein X is a bond, —O—, —S— —S(O)—, —SO₂—, or —NH—, Z is —O—, —S—, —(C═O)—, —(C═S)—, —S(O)—, —SO₂—, —NR^{A}, or, in either orientation —C(═O)O—, —C(═O)NR^{A}, —C(═S)NR^{A}—, —SO₂NR^{A}—, —NR^{A}C(═O)—, or —NR^{A}SO₂— wherein R^{A} is hydrogen or C₁-C₆ alkyl in which one or more hydrogens is optionally substituted by fluorine; Alk¹ and Alk² are optionally substituted divalent C₁-C₃ alkylene or C₂-C₃ alkenylene radicals, m and n are independently 0 or 1, and Q is hydrogen or an optionally substituted carbocyclic or heterocyclic radical; R₂ is cyano (—CN), fluoro, chloro, bromo, methyl, ethyl, —OH, —CH₂OH, —C(═O)NH₂, —C(═O)H, —C(═O)CH₃, or —NH₂; R₃ and R₄ are independently selected from hydrogen, fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or R₃ and R₄ together represent a carbocyclic or heterocyclic ring fused to ring A, or methylenedioxy (—OCH₂O—) or ethylenedioxy (—OCH₂CH₂O—) in either of which one or more hydrogens are optionally replaced by fluorine; S₁ is hydrogen, or a substituent as defined in the specification.

This invention relates to substituted purine compounds having HSP90 inhibitory activity, to the use of such compounds in medicine, in relation to diseases which are responsive to inhibition of HSP90 activity such as cancers, and to pharmaceutical compositions containing such compounds.

### BACKGROUND TO THE INVENTION

Molecular chaperones maintain the appropriate folding and conformation of proteins and are crucial in regulating the balance between protein synthesis and degradation. They have been shown to be important in regulating many important cellular functions, such as cell proliferation and apoptosis (Jolly and Morimoto, 2000; Smith et al., 1998; Smith, 2001).

### Heat Shock Proteins (HSPs)

Exposure of cells to a number of environmental stresses, including heat shock, alcohols, heavy metals and oxidative stress, results in the cellular accumulation of a number of chaperones, commonly known as heat shock proteins (HSPs). Induction of HSPs protects the cell against the initial stress insult, enhances recovery and leads to maintenance of a stress tolerant state. It has also become clear, however, that certain HSPs may also play a major molecular chaperone role under normal, stress-free conditions by regulating the correct folding, degradation, localization and function of a growing list of important cellular proteins.

A number of multigene families of HSPs exist, with individual gene products varying in cellular expression, function and localization. They are classified according to molecular weight, e.g., HSP70, HSP90, and HSP27. Several diseases in humans can be acquired as a result of protein misfolding (reviewed in Tytell et al., 2001; Smith et al., 1998). Hence the development of therapies which disrupt the molecular chaperone machinery may prove to be beneficial. In some conditions (e.g., Alzheimer's disease, prion diseases and Huntington's disease), misfolded proteins can cause protein aggregation resulting in neurodegenerative disorders. Also, misfolded proteins may result in loss of wild type protein function, leading to deregulated molecular and physiological functions in the cell.

HSPs have also been implicated in cancer. For example, there is evidence of differential expression of HSPs which may relate to the stage of tumour progression (Martin et al., 2000; Conroy et al., 1996; Kawanishi et al., 1999; Jameel et al., 1992; Hoang et al., 2000; Lebeau et al., 1991). As a result of the involvement of HSP90 in various critical oncogenic pathways and the discovery that certain natural products with anticancer activity are targeting this molecular chaperone, the fascinating new concept has been developed that inhibiting HSP function may be useful in the treatment of cancer. The first molecular chaperone inhibitor is currently undergoing clinical trials.

### HSP90

HSP90 constitutes about 1-2% of total cellular protein, and is usually present in the cell as a dimer in association with one of a number of other proteins (see, e.g., Pratt, 1997). It is essential for cell viability and it exhibits dual chaperone functions (Young et al., 2001). It plays a key role in the cellular stress response by interacting with many proteins after their native conformation has been altered by various environmental stresses, such as heat shock, ensuring adequate protein folding and preventing non-specific aggregation (Smith et al., 1998). In addition, recent results suggest that HSP90 may also play a role in buffering against the effects of mutation, presumably by correcting the inappropriate folding of mutant proteins (Rutherford and Lindquist, 1998). However, HSP90 also has an important regulatory role. Under normal physiological conditions, together with its endoplasmic reticulum homologue GRP94, HSP90 plays a housekeeping role in the cell, maintaining the conformational stability and maturation of several key client proteins. These can be subdivided into three groups: (a) steroid hormone receptors, (b) Ser/Thr or tyrosine kinases (e.g., ERBB2, RAF-1, CDK4, and LCK), and (c) a collection of apparently unrelated proteins, e.g., mutant p53 and the catalytic subunit of telomerase hTERT. All of these proteins play key regulatory roles in many physiological and biochemical processes in the cell. New HSP90 client proteins are continuously being identified.

The highly conserved HSP90 family in humans consists of four genes, namely the cytosolic HSP90α and HSP90β isoforms (Hickey et al., 1989), GRP94 in the endoplasmic reticulum (Argon et al., 1999) and HSP75/TRAP1 in the mitochondrial matrix (Felts et al., 2000). It is thought that all the family members have a similar mode of action, but bind to different client proteins depending on their localization within the cell. For example, ERBB2 is known to be a specific client protein of GRP94 (Argon et al., 1999) and type 1 tumour necrosis factor receptor (TNFR1) and RB have both been shown to be clients of TRAP1 (Song et al., 1995; Chen et al., 1996).

HSP90 participates in a series of complex interactions with a range of client and regulatory proteins (Smith, 2001). Although the precise molecular details remain to be elucidated, biochemical and X-ray crystallographic studies (Prodromou et al., 1997; Stebbins et al., 1997) carried out over the last few years have provided increasingly detailed insights into the chaperone function of HSP90.

Following earlier controversy on this issue, it is now clear that HSP90 is an ATP-dependent molecular chaperone (Prodromou et al, 1997), with dimerization of the nucleotide binding domains being essential for ATP hydrolysis, which is in turn essential for chaperone function (Prodromou et al, 2000a). Binding of ATP results in the formation of a toroidal dimer structure in which the N terminal domains are brought into closer contact with each other resulting in a conformational switch known as the ‘clamp mechanism’ (Prodromou and Pearl, 2000b).

### Known HSP90 Inhibitors

The first class of HSP90 inhibitors to be discovered was the benzoquinone ansamycin class, which includes the compounds herbimycin A and geldanamycin. They were shown to reverse the malignant phenotype of fibroblasts transformed by the v-Src oncogene (Uehara et al., 1985), and subsequently to exhibit potent antitumour activity in both in vitro (Schulte et al., 1998) and in vivo animal models (Supko et al., 1995).

Immunoprecipitation and affinity matrix studies have shown that the major mechanism of action of geldanamycin involves binding to HSP90 (Whitesell et al., 1994; Schulte and Neckers, 1998). Moreover, X-ray crystallographic studies have shown that geldanamycin competes at the ATP binding site and inhibits the intrinsic ATPase activity of HSP90 (Prodromou et al., 1997; Panaretou et al., 1998). This in turn prevents the formation of mature multimeric HSP90 complexes capable of chaperoning client proteins. As a result, the client proteins are targeted for degradation via the ubiquitin proteasome pathway. 17-Allylamino, 17-demethoxygeldanamycin (17AAG) retains the property of HSP90 inhibition resulting in client protein depletion and antitumour activity in cell culture and xenograft models (Schulte et al, 1998; Kelland et al, 1999), but has significantly less hepatotoxicity than geldanamycin (Page et al, 1997). 17AAG is currently being evaluated in Phase I clinical trials.

Radicicol is a macrocyclic antibiotic shown to reverse the malignant phenotype of v-Src and v-Ha-Ras transformed fibroblasts (Kwon et al, 1992; Zhao et al, 1995). It was shown to degrade a number of signalling proteins as a consequence of HSP90 inhibition (Schulte et al., 1998). X-ray crystallographic data confirmed that radicicol also binds to the N terminal domain of HSP90 and inhibits the intrinsic ATPase activity (Roe et al., 1998). Radicicol lacks antitumour activity in vivo due to the unstable chemical nature of the compound.

Coumarin antibiotics are known to bind to bacterial DNA gyrase at an ATP binding site homologous to that of the HSP90. The coumarin, novobiocin, was shown to bind to the carboxy terminus of HSP90, i.e., at a different site to that occupied by the benzoquinone ansamycins and radicicol which bind at the N-terminus (Marcu et al., 2000b). However, this still resulted in inhibition of HSP90 function and degradation of a number of HSP90-chaperoned signalling proteins (Marcu et al., 2000a). Geldanamcyin cannot bind HSP90 subsequent to novobiocin; this suggests that some interaction between the N and C terminal domains must exist and is consistent with the view that both sites are important for HSP90 chaperone properties.

A purine-based HSP90 inhibitor, PU3, has been shown to result in the degradation of signalling molecules, including ERBB2, and to cause cell cycle arrest and differentiation in breast cancer cells (Chiosis et al., 2001).

Patent publications WO 2004/050087 and WO 2004/056782 relate to known classes pyrazole derivatives which are HSP90 inhibitors.

### HSP90 as a Therapeutic Target

Due to its involvement in regulating a number of signalling pathways that are crucially important in driving the phenotype of a tumour, and the discovery that certain bioactive natural products exert their effects via HSP90 activity, the molecular chaperone HSP90 is currently being assessed as a new target for anticancer drug development (Neckers et al., 1999).

The predominant mechanism of action of geldanamycin, 17AAG, and radicicol involves binding to HSP90 at the ATP binding site located in the N-terminal domain of the protein, leading to inhibition of the intrinsic ATPase activity of HSP90 (see, e.g., Prodromou et al., 1997; Stebbins et al., 1997; Panaretou et al., 1998).

Inhibition of HSP90 ATPase activity prevents recruitment of co-chaperones and encourages the formation of a type of HSP90 heterocomplex from which these client proteins are targeted for degradation via the ubiquitin proteasome pathway (see, e.g., Neckers et al., 1999; Kelland et al., 1999).

Treatment with HSP90 inhibitors leads to selective degradation of important proteins involved in cell proliferation, cell cycle regulation and apoptosis, processes which are fundamentally important in cancer.

Inhibition of HSP90 function has been shown to cause selective degradation of important signalling proteins involved in cell proliferation, cell cycle regulation and apoptosis, processes which are fundamentally important and which are commonly deregulated in cancer (see, e.g., Hostein et al., 2001). An attractive rationale for developing drugs against this target for use in the clinic is that by simultaneously depleting proteins associated with the transformed phenotype, one may obtain a strong antitumour effect and achieve a therapeutic advantage against cancer versus normal cells. These events downstream of HSP90 inhibition are believed to be responsible for the antitumour activity of HSP90 inhibitors in cell culture and animal models (see, e.g., Schulte et al., 1998; Kelland et al., 1999).

Hsp90 inhibitors can resensitise previously resistant fungal strains to the commonly used azole antifungal agents (e.g. fluconazole) as well as newer agents such as echinocandins (see Cowen and Lindquist, Science, Vol 309, 30 Sep. 2005, 2185-2189.)

### BRIEF DESCRIPTION OF THE INVENTION

This invention is based on the finding that a class of aryl- or heteroaryl-substituted purine compounds has Hsp90 inhibitory activity, and is of interest in the treatment of diseases responsive to inhibition of Hsp90 activity.

Patent publication WO 2006/046023 is concerned with ortho-condensed pyridine and pyrimidine derivatives (eg purines) as protein kinase inhibitors. The definition of the compounds with which that publication is concerned is very broad, and includes compounds having a purine scaffold. However, since the publication is concerned with protein kinase inhibitors, it provides no information concerning the activity of 4-aryl or 4-heteroaryl purine derivatives against Hsp90.

### DETAILED DESCRIPTION OF THE INVENTION

In one broad aspect the present invention provides compound of formula (I), or a salt, N-oxide, hydrate, or solvate thereof:

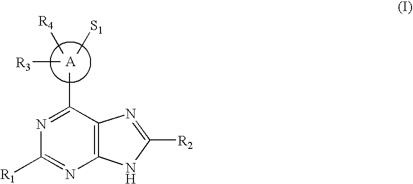

wherein
ring A is an aryl or heteroaryl ring or ring system;
R₁ is hydrogen, fluoro, chloro, bromo, or a radical of formula (1A):

—X-Alk¹-(Z)ₘ-(Alk²)ₙ-Q (IA)

wherein
X is a bond, —O—, —S— —S(O)—, —SO₂—, or —NH—,
Z is —O—, —S—, —(C═O)—, —(C═S)—, —S(O)—, —SO₂—, —NR^{A}, or, in either orientation —C(═O)O— —C(═O)NR^{A}—, —C(═S)NR^{A}—, —SO₂NR^{A}—, —NR^{A}C(═O)—, or —NR^{A}SO₂— wherein R^{A} is hydrogen or C₁-C₆ alkyl in which one or more hydrogens is optionally substituted by fluorine;
Alk¹ and Alk² are optionally substituted divalent C₁-C₃ alkylene or C₂-C₃ alkenylene radicals,
m and n are independently 0 or 1, and
Q is hydrogen or an optionally substituted carbocyclic or heterocyclic radical;
   R₂ is cyano (—CN), fluoro, chloro, bromo, methyl, ethyl, —OH, —CH₂OH, —C(═O)NH₂, —C(═O)H, —C(═O)CH₃, or —NH₂;
   R₃ and R₄ are independently selected from hydrogen, fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or R₃ and R₄ together represent a carbocyclic or heterocyclic ring fused to ring A, or methylenedioxy (—OCH₂O—) or ethylenedioxy (—OCH₂CH₂O—) in either of which one or more hydrogens are optionally replaced by fluorine;
   S₁ is hydrogen, or a substituent selected from fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or S₁ and R₃, or S₁ and R₄, together represent methylenedioxy (—OCH₂O—) or ethylenedioxy ((—OCH₂ CH₂O—) in either of which one or more hydrogens are optionally replaced by fluorine; or S₁ is a radical of formula (IB):

-(Alk³)ₚ-(Z¹)_{q}-(Alk⁴)ᵣ-Q¹ (IB)

wherein
p, q and r are independently 0 or 1;
(a) when p is 0 or 1, and q is 1, and r is 0 or 1:
Z¹ is selected from the group of divalent radicals consisting of (i) —S—, —(C═O)—,
—(C═S)—, —S(O)— and —SO₂— and (ii) —N(R^{A})C(═O)—* wherein the bond marked * is attached to Q¹ and (iii) in either orientation, —C(═O)O—, —C(═S)NR^{A}—, and —SO₂NR^{A}—; and Q¹ is (i) hydrogen or an optional substituent; or (ii) an optionally substituted carbocyclic or heterocyclic radical; or (iii) a radical —CH₂[O(CH₂)_{w}]_{z}Z² wherein Z² is H, —OH or —O(C₁-C₃alkyl) wherein x and w are independently 1, 2 or 3; or
(b) when p is 1, and q is 1, and r is 0 or 1:
Z¹ is —O—, and Q¹ is (i) hydrogen or an optional substituent which is not linked to -(Alk³)ₚ-(Z¹)_{q}-(Alk⁴)ᵣ— through a nitrogen atom; or (ii) an optionally substituted carbocyclic radical; or (iii) an optionally substituted heterocyclic ring of 5 or 6 ring atoms which is not linked to -(Alk³)ₚ-(Z¹)_{q}-(Alk⁴)ᵣ— through a ring nitrogen; or (iv) a radical —CH₂[O(CH₂)_{w}]ₓZ² wherein Z² is H, —OH or —O(C₁-C₃alkyl) wherein x and w are independently 1, 2 or 3. or
(c) when p is 1, and q is 1, and r is 0 or 1:
Z¹ is —NR^{A}— or —C(═O)N(R^{A})—* wherein the bond marked * is attached to Q¹ and Q¹ is a radical —CH₂[O(CH₂)_{w}]ₓZ² wherein Z² is H, —OH or —O(C₁-C₃alkyl) wherein x and w are independently 1, 2 or 3. or
(d) when p is 0, and q is 1, and r is 0 or 1:
Z¹ is —O— or —NR^{A}— and Q¹ is (i) hydrogen or an optional substituent which is not linked to -(Alk³)ₚ-(Z¹)_{q}-(Alk⁴)ᵣ— through a nitrogen atom; or (ii) Q¹ and R^{A}, taken together with the nitrogen to which they are attached form an optionally substituted heterocyclic ring of 5 or 6 ring atoms; or (iii) a radical —CH₂[O(CH₂)_{w}]ₓZ² wherein Z² is H, —OH or —O(C₁-C₃alkyl) wherein x and w are independently 1, 2 or 3; or
(e) when p is 0 or 1, q is 0, and r is 0 or 1:
Q¹ is (i) hydrogen or an optional substituent which is not linked to -(Alk³)ₚ-(Z¹)_{q}-(Alk⁴)ᵣ— through a nitrogen atom or (ii) an optionally substituted carbocyclic radical; or (iii) an optionally substituted heterocyclic of 5 or 6 ring atoms which is not linked to -(Alk³)ₚ-(Z¹)_{q}-(Alk⁴)ᵣ— through a ring nitrogen; or (iv) a radical —CH₂[O(CH₂)_{w}]ₓZ² wherein Z² is H, —OH or —O(C₁-C₃alkyl) wherein x and w are independently 1, 2 or 3;
R^{A} is hydrogen or C₁-C₃ alkyl optionally substituted with one or more fluorine substituents; and
Alk³ and Alk⁴ are divalent C₁-C₃ alkylene or C₂-C₃ alkenylene radicals, each optionally substituted by one or two substituents selected from fluoro, chloro, C₁-C₃alkyl optionally substituted with one or more fluorine substituents, C₁-C₃alkoxy optionally substituted with one or more fluorine substituents.

Compounds of formula (I) above are tautomeric with compounds of formula (II):

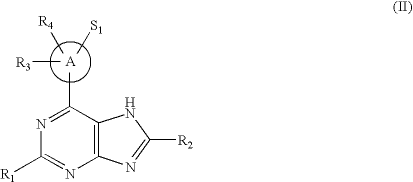

The present invention includes compounds of either tautomeric form and mixtures thereof. References herein to compounds having the purine ring structure shown formula (I) are to be taken as including compounds having the purine ring structure shown in (II), and mixtures thereof.

Compounds of the invention include those of formula (I) wherein:ring A is a phenyl ring; and R₂ is hydrogen; and, in the substituent R₁, X is a bond, and p is 1, and Z¹ is —O—, —S—, —(C═O)—, —(C═S)—, —SO₂—, —C(═O)O—, —C(═O)NR^{A}—, —C(═S)NR^{A}—, —SO₂NR^{A}—, —NR^{A}C(═O)—, —NR^{A}SO₂— or —NR^{A}— wherein R^{A} is hydrogen or C₁-C₆ alkyl; and in the phenyl ring A:
S₁ is a radical of formula (IB) wherein: p is 0 or 1, and q is 1, and r is 0 or 1, Z¹ is selected from the group of divalent radicals consisting of (i) —S—, —(C═O)—, —(C═S)—, and —SO₂— and (ii) —N(R^{A})C(═O)—* wherein the bond marked * is attached to Q¹ and (iii) in either orientation, —C(═O)O—, —C(═S)NR^{A}— and —SO₂NR^{A}—; and Q¹ is (i) hydrogen or (ii) an optionally substituted carbocyclic or heterocyclic radical, or
S₁ is a radical of formula (IB) wherein p is 1, and q is 1, and r is 0 or 1, Z¹ is —O—, and Q¹ is (i) hydrogen or (ii) an optionally substituted carbocyclic radical; or (iii) an optionally substituted heterocyclic ring of 5 or 6 ring atoms which is not linked to -(Alk³)ₚ-(Z¹)_{q}-(Alk⁴)ᵣ— through a ring nitrogen, or
S₁ is a radical of formula (IB) wherein p is 0 or 1, q is 0, and r is 0 or 1, and Q¹ is (i) hydrogen or (ii) an optionally substituted carbocyclic radical; or (iii) an optionally substituted heterocyclic of 5 or 6 ring atoms which is not linked to -(Alk³)ₚ-(Z¹)_{q}-(Alk⁴)ᵣ— through a ring nitrogen.

The invention also comprises the use of a compound of formula (I) above in the preparation of a composition for inhibition of HSP90 activity in vitro or in vivo.

In another broad aspect, the invention provides a method of treatment of diseases which are responsive to inhibition of HSP90 activity in mammals, which method comprises administering to the mammal an amount of a compound of formula (I) above effective to inhibit said HSP90 activity.

The in vivo use, and method, of the invention is applicable to the treatment of diseases in which HSP90 activity is implicated, including use for immunosuppression or the treatment of viral disease, inflammatory diseases such as rheumatoid arthritis, asthma, multiple sclerosis, Type I diabetes, lupus, psoriasis and inflammatory bowel disease; cystic fibrosis angiogenesis-related disease such as diabetic retinopathy, haemangiomas, and endometriosis; or for protection of normal cells against chemotherapy-induced toxicity; or diseases where failure to undergo apoptosis is an underlying factor; or protection from hypoxia-ischemic injury due to elevation of Hsp70 in the heart and brain; scrapie/CJD, Huntingdon's or Alzheimer's disease. Use as co-therapy with antifungal drugs in the treatment of drug resistant fangal infections is also indicated. Use for the treatment of cancer is especially indicated.

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “divalent (Cₐ-C_{b})alkylene radical” wherein a and b are integers refers to a saturated hydrocarbon chain having from a to b carbon atoms and two unsatisfied valences.

As used herein the term “(Cₐ-C_{b})alkenyl” wherein a and b are integers refers to a straight or branched chain alkenyl moiety having from a to b carbon atoms having at least one double bond of either E or Z stereochemistry where applicable. The term includes, for example, vinyl, allyl, 1- and 2-butenyl and 2-methyl-2-propenyl.

As used herein the term “divalent (Cₐ-C_{b})alkenylene radical” refers to a hydrocarbon chain having from a to b carbon atoms, at least one double bond, and two unsatisfied valences.

As used herein the term “cycloalkyl” refers to a saturated carbocyclic radical having from 3-8 carbon atoms and includes, for example, cyclopropyl, cyclobutyl, cyclopentyl, cyclohexyl, cycloheptyl and cyclooctyl.

As used herein the term “cycloalkenyl” refers to a carbocyclic radical having from 3-8 carbon atoms containing at least one double bond, and includes, for example, cyclopentenyl, cyclohexenyl, cycloheptenyl and cyclooctenyl.

As used herein the term “aryl” refers to a mono-, bi- or tri-cyclic carbocyclic aromatic radical. Illustrative of such radicals are phenyl, biphenyl and napthyl.

As used herein the term “carbocyclic” refers to a cyclic radical whose ring atoms are all carbon, and includes monocyclic aryl, cycloalkyl, and cycloalkenyl radicals.

As used herein the term “heteroaryl” refers to a mono-, bi- or tri-cyclic aromatic radical containing one or more heteroatoms selected from S, N and O. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in particular refers to a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

Unless otherwise specified in the context in which it occurs, the term “substituted” as applied to any moiety herein means substituted with at least one substituent, for example selected from (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, monocyclic carbocyclic of 3-6 ring carbon atoms, monocyclic heterocyclic of 5 or 6 ring atoms, halo (including fluoro and chloro), trifluoromethyl, trifluoromethoxy, nitro, nitrile (—CN), oxo, —COOH, —COOR^{A}, —COR^{A} —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHCOOR^{A}, —NR^{B}COOR^{A}, —NHSO₂OR^{A}, —NR^{B}SO₂OR^{A}, —NHCONH₂, —NR^{A}CONH₂, —NHCONHR^{B}, —NR^{A}CONHR^{B}, —NHCONR^{A}R^{B} or —NR^{A}CONR^{A}R^{B} wherein R^{A} and R^{B} are independently a (C₁-C₆)alkyl group in which one or more nitrogens are optionally replaced by fluorine or R^{A} and R^{B} when attached to the same nitrogen may form, together with that nitrogen, a cyclic amino ring such as a morpholinyl, piperidinyl, piperazinyl, N-methyl piperazinyl, pyrrolidinyl or 2-oxo-pyrrolidinyl ring. In the case where the optional substituent contains an alkyl radical, that alkyl radical may be substituted by one or more fluorines, and/or by a monocyclic carbocyclic group of 3-6 ring carbon atoms, or a monocyclic heterocyclic group of 5 or 6 ring atoms. In the case where the optional substituent is or comprises a monocyclic carbocyclic group of 3-6 ring carbon atoms, or a monocyclic heterocyclic group of 5 or 6 ring atoms, that ring may itself be substituted by any of the non-cyclic optional substituents listed above. An “optional substituent” may be one of the substituent groups encompassed in the above description.

As used herein the term “salt” includes base addition, acid addition and quaternary salts. Compounds of the invention which are acidic can form salts, including pharmaceutically or veterinarily acceptable salts, with bases such as alkali metal hydroxides, e.g. sodium and potassium hydroxides; alkaline earth metal hydroxides e.g. calcium, barium and magnesium hydroxides; with organic bases e.g. N-ethyl piperidine, dibenzylamine and the like. Those compounds (I) which are basic can form salts, including pharmaceutically or veterinarily acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic and p-toluene sulphonic acids and the like.

For a review on suitable salts, see Handbook of Pharmaceutical Salts: Properties, Selection, and Use by Stahl and Wermuth (Wiley-VCH, Weinheim, Germany, 2002).

The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Compounds with which the invention is concerned which may exist in one or more stereoisomeric form, because of the presence of asymmetric atoms or rotational restrictions, can exist as a number of stereoisomers with R or S stereochemistry at each chiral centre or as atropisomeres with R or S stereochemistry at each chiral axis. The invention includes all such enantiomers and diastereoisomers and mixtures thereof.

So-called ‘pro-drugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985).

Also included within the scope of the invention are metabolites of compounds of formula (I), that is, compounds formed in vivo upon administration of the drug. Some examples of metabolites include
(i) where the compound of formula (I) contains a methyl group, an hydroxymethyl derivative thereof (—CH₃->—CH₂OH):
(ii) where the compound of formula (I) contains an alkoxy group, an hydroxy derivative thereof (—OR->—OH);
   (iii) where the compound of formula (I) contains a tertiary amino group, a secondary amino derivative thereof (—NR¹R²—>—NHR¹ or —NHR²);
   (iv) where the compound of formula (I) contains a secondary amino group, a primary derivative thereof (—NHR¹->—NH₂);
   (v) where the compound of formula (I) contains a phenyl moiety, a phenol derivative thereof (-Ph->-PhOH); and
   (vi) where the compound of formula (I) contains an amide group, a carboxylic acid derivative thereof (—CONH₂->COOH).
      The group R₁

When R₁ is a radical of formula (1A):

—X-Alk¹-(Z)ₘ-(Alk²)ₙ-Q (IA)
X may be —O—, —S— —S(O)—, —SO₂—, or —NH—. At present —O— and —S— are preferred;
when present, Z may be —O—, —S—, —(C═O)—, —(C═S)—, —S(O)—, —SO₂—, —NR^{A}—, or, in either orientation —C(═O)O—, —C(═O)NR^{A}—, —C(═S)NR^{A}—, —SO₂NR^{A}—, —NR^{A}C(═O)—, or —NR^{A}SO₂— wherein R^{A} is hydrogen or C₁-C₆ alkyl. At present —NR^{A}— is preferred;
Alk¹ (and Alk² when present) may be, for example —CH₂—, —CH₂CH₂—, —CH₂CH₂CH₂—, —CH(CH₃)CH₂— or —CH₂CH═CH—;
m and n are independently 0 or 1. Thus, in one class of radicals (IA), m and n are both 0. In another class of radicals (IA), m is 1 and n is 0. In a further class of radicals (IA), m is 0 and n is 1;
Q may be hydrogen or an optionally substituted carbocyclic or heterocyclic radical. Examples of carbocyclic radicals Q include phenyl, cyclopropyl, cyclobutyl, cyclopentyl and cyclohexyl. Examples of heterocyclic radicals Q include heteroaryl radicals such as pyridyl, thienyl and furanyl, and non-aromatic heterocyclic radicals such as piperidinyl, piperazinyl and morpholinyl.
Currently it is preferred that Alk¹, Alk₂ and Q (when carbocyclic or heterocyclic) are unsubstituted. However, examples of substituents which may be present in Alk¹, Alk₂ and Q (when carbocyclic or heterocyclic) include methyl, ethyl, n- or isopropyl, vinyl, allyl, methoxy, ethoxy, n-propyloxy, isopropyloxy, benzyloxy, allyloxy, cyanomethoxy chloro, bromo, cyano, formyl, methyl-, ethyl-, or n-propyl-carbonyloxy, methyl- or ethylaminocarbonyl, and substituents of formula —O(CH₂)₂Z¹ wherein a is 1, 2 or 3 and Z¹ is a primary, secondary, tertiary or cyclic amino group, or a C₁-C₆alkoxy group; or of formula -(Alk³)_{b}Z¹ wherein Alk³ is a divalent straight or branched chain (C₁-C₃) alkylene, b is 0 or 1, and Z¹ is a primary, secondary, tertiary or cyclic amino group, or a C₁-C₆alkoxy group.

In one class of compounds of the invention, R₁ is a radical of formula —W-Alk⁵-B wherein W is —O— or —S—, Alk⁵ is a straight or branched divalent C₁-C₆ alkylene radical in which one or more hydrogen atoms is/are replaced by fluorine atoms, and B is hydrogen, —NH₂, —NHR^{A}, NHR^{A}R^{B} wherein R^{A} and R^{B} are independently hydrogen or C₁-C₆ alkyl or C₁-C₆ alkyl in which one or more hydrogen atoms is/are replaced by fluorine atoms, or R^{A} and R^{B} together with the nitrogen to which they are attached form a saturated 5- or 6-membered heterocyclic ring. For example, -Alk⁵- may be for example —CH₂CH₂— or —CH₂ CH₂CH₂—. Also by way of example, B may be ethylamino, diethylamino, methylamino, dimethylamino, morpholinyl, piperidinyl, piperazinyl, N-methyl piperazinyl, pyrrolidinyl or 2-oxo-pyrrolidinyl. In cases where B is hydrogen, examples of R₁ include methoxy, ethoxy, methylthio or ethylthio,

### The Group R₂

At present, it is preferred that R₂ is hydrogen or cyano (—CN).

### The Ring A

Ring A is an aryl or heteroaryl ring or ring system, for example phenyl, thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl or indazolyl. Currently it is preferred that ring A is phenyl.

### The Groups R₃ and R₄

R₃ and R₄ are independently selected from hydrogen, fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or R₃ and R₄ together represent a carbocyclic or heterocyclic ring fused to ring A, for example a benz-fused ring, methylenedioxy (—OCH₂O—) or ethylenedioxy (—OCH₂CH₂O—) in either of which one or more hydrogens are optionally replaced by fluorine. However at least one of R₃ and R₄ should preferably be other than hydrogen. Presently preferred is the case where one or both of R₃ and R₄ is/are selected from fluoro, chloro, methyl or methoxy. Preferred positions for R₃ and R₄ when ring A is phenyl are the para and ortho positions.

### The Group S₁

When S₁ is other than hydrogen, and ring A is phenyl, it is presently preferred that S₁ be in the meta position of the ring.

In a first subset of compounds (I) of the invention, S₁ is hydrogen, or a substituent selected from fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or S₁ and R₃, or S₁ and R₄, together represent methylenedioxy (—OCH₂O—) or ethylenedioxy (—OCH₂CH₂O—) in either of which one or more hydrogens are optionally replaced by fluorine;

In a second subset of compounds (I) of the invention, S₁ is a radical of formula (IB):

-(Alk³)ₚ-(Z¹)_{q}-(Alk⁴)ᵣ-Q¹ (IB)

wherein p, q, r, Alk³, Alk⁴, Z¹ and Q¹ are as defined in relation to formula (I) above. In such compounds, Alk³ and Alk⁴ are divalent C₁-C₃ alkylene or C₂-C₃ alkenylene radicals, each optionally substituted by one or two substituents selected from fluoro, chloro, C₁-C₃alkyl optionally substituted with one or more fluorine substituents, C₁-C₃alkoxy optionally substituted with one or more fluorine substituents. Examples of radicals Alk³ and Alk⁴ when present, are example —CH₂— —CH₂CH₂— —CH₂CH₂CH₂—CH(CH₃)CH₂—, CH₂CH(CH₃)CH₂—, —CH₂CH═CH—; —CH(OCH₃)CH₂—, and —CH₂CH(OCH₃)CH₂—,

In this second subset of compounds (I) of the invention there are five specific combinations (a)-(e) of p, q, r, Alk³, Alk⁴, Z¹ and Q¹:

Case (a) arises when p is 0 or 1, and q is 1, and r is 0 or 1. In case (a), Z¹ is selected from the group of divalent radicals consisting of (i) —S—, —(C═O)—, —(C═S)—, —S(O)— and —SO₂— and (ii) —N(R^{A})C(═O)—* wherein the bond marked * is attached to Q¹ and (iii) in either orientation, —C(═O)O—, —C(═S)NR^{A}—, and —SO₂NR^{A}—; and Q¹ is (i) hydrogen or an optional substituent; or (ii) an optionally substituted carbocyclic or heterocyclic radical; or (iii) a radical —CH₂[O(CH₂)_{w}]ₓZ² wherein Z² is H, —OH or —O(C₁-C₃alkyl) wherein x and w are independently 1, 2 or 3.

R^{A} when present in Z¹ and when other than hydrogen may be, for example, methyl, ethyl, n- or iso-propyl, or trifluoromethyl.

When other than hydrogen, Q¹ may be, for example
an primary, secondary or tertiary amino substituent, for example —NR^{A}R^{B} wherein R^{A} and R^{B} are independently selected from hydrogen and C₁-C₃alkyl in which one or more hydrogens is optionally replaced by fluorine, for example methylamino, dimethylamino, ethylamino, diethylamino, n- or iso-propylamino, or N-methyl-N-ethylamino and N-(1,1,1-trifluoroethyl)-N-ethylamino,
a non-amino optional substituent, for example chloro, C₁-C₃alkoxy, cyano or acetyl; or a cyclopropyl, cylopenyl or cyclohexyl group;
an optionally substituted phenyl group, for example wherein optional substituents are selected from cyano (—CN), fluoro, chloro, bromo, methyl, ethyl, —OH, —CH₂OH, —C(═O)NH₂, —C(═O)H, —C(═O)CH₃, and —NH₂;
a cyclic amino group such as morpholino, piperidinyl, piperazinyl or methylpiperidinyl or a fluoro substituted cyclic amino group such as those of formulae (A)-(D):

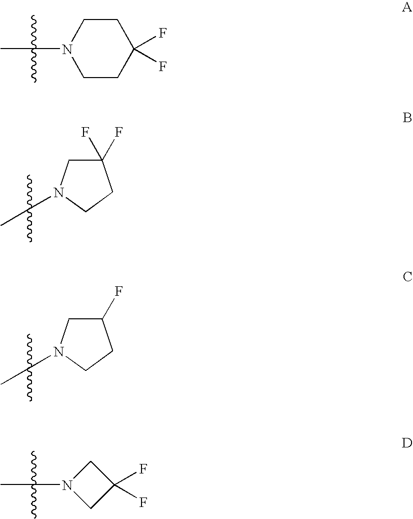

a saturated carbocylic group such as cyclopropyl, cyclopentyl, cyclohexyl or norbornyl;
a heterocyclic group such as any of those heteroaryl groups referred to above as examples of ring A, or a non aromatic heterocyclic group such as one having the formula E:

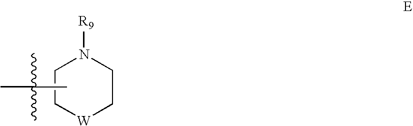

wherein W is —CH₂—, —O—, —S— or —NR₉, and R₉ is hydrogen, methyl, ethyl or n- or iso-propyl; or
a radical —CH₂[O(CH₂)_{w}]ₓZ² wherein Z² is H, —OH or —OCH₃ wherein x and w are independently 1, 2 or 3. Such radicals include the polyether radicals —O—(CH₂)₁₋₃OH, —O—(CH₂)₁₋₃O(C₁-C₃alkyl)-O—(CH₂)₁₋₃—O—(CH₂)₁₋₃OH, and —O—(CH₂)₁₋₃—O—(CH₂)₁₋₃O (C₁-C₃alkyl),

Case (b) arises when p is 1, and q is 1, and r is 0 or 1 and Z¹ is —O—. In case (b) Q¹ is (i) hydrogen or an optional substituent which is not linked to -(Alk³)ₚ-(Z¹)_{q}-(Alk⁴)ᵣ-through a nitrogen atom; or (ii) an optionally substituted carbocyclic radical; or (iii) an optionally substituted heterocyclic ring of 5 or 6 ring atoms which is not linked to -(Alk³)ₚ-(Z¹)_{q}-(Alk⁴)ᵣ— through a ring nitrogen; or (iv) a radical —CH₂[O(CH₂)_{w}]ₓZ² wherein Z² is H, —OH or —O(C₁-C₃alkyl) wherein x and w are independently 1, 2 or 3.

In this case (b), when other than hydrogen, Q¹ may be, for example
a non-amino optional substituent, for example chloro, C₁-C₃alkoxy, cyano or acetyl; or a cyclopropyl, cylopenyl or cyclohexyl group;
an optionally substituted phenyl group, for example wherein optional substituents are selected from cyano (—CN), fluoro, chloro, bromo, methyl, ethyl, —OH, —CH₂OH, —C(═O)NH₂, —C(═O)H, —C(═O)CH₃, and —NH₂;
a saturated carbocylic group such as cyclopropyl, cyclopentyl, cyclohexyl or norbornyl;
a heterocyclic group such as any of those heteroaryl groups referred to above as examples of ring A, or a non aromatic heterocyclic group such as one having formula E defined above; or
a radical —CH₂[O(CH₂)_{w}]ₓZ² wherein Z² is H, —OH or —OCH₃ wherein x and w are independently 1, 2 or 3. Such radicals include the polyether radicals —O—(CH₂)₁₋₃OH, —O—(CH₂)₁₋₃O(C₁-C₃alkyl)-O—(CH₂)₁₋₃—O—(CH₂)₁₋₃OH, and —O—(CH₂)₁₋₃—O—(CH₂)₁₋₃O(C₁-C₃alkyl).

Case (c) arises when p is 1, and q is 1, and r is 0 or 1 and Z¹ is —NR^{A}— or —C(═O)N(R^{A})—* wherein the bond marked * is attached to Q¹. In this case (c):
R^{A} when other than hydrogen may be, for example, methyl, ethyl, n- or iso-propyl, or trifluoromethyl; and
Q¹ is a radical —CH₂[O(CH₂)_{w}]ₓZ² wherein Z² is H, —OH or —O(C₁-C₃alkyl) wherein x and w are independently 1, 2 or 3. Such radicals include the polyether radicals —O—(CH₂)₁₋₃OH, —O—(CH₂)₁₋₃O(C₁-C₃alkyl), —O—(CH₂)₁₋₃—O—(CH₂)₁₋₃OH, and —O—(CH₂)₁₋₃—O—(CH₂)₁₋₃O(C₁-C₃alkyl).

Case (d) arises when p is 0, and q is 1, and r is 0 or 1 and Z¹ is —O— or —NR^{A}—. In this case (d) Q¹ is (i) hydrogen or an optional substituent which is not linked to -(Alk³)ₚ-(Z¹)_{q}-(Alk⁴)ᵣ- through a nitrogen atom; or (ii) Q¹ and R^{A}, taken together with the nitrogen to which they are attached form an optionally substituted heterocyclic ring of 5 or 6 ring atoms; or (iii) a radical —CH₂[O(CH₂)_{w}]ₓZ² wherein Z² is H, —OH or —OCH₃ wherein x and w are independently 1, 2 or 3. In this case (d) R^{A} when other than hydrogen may be, for example, methyl, ethyl, n- or iso-propyl, or trifluoromethyl; and Q¹ may be, for example:
a non-amino optional substituent, for example chloro, C₁-C₃alkoxy, cyano or acetyl; or a cyclopropyl, cylopentyl or cyclohexyl group;
a radical —CH₂[O(CH₂)_{w}]ₓZ² wherein Z² is H, —OH or —OCH₃ wherein x and w are independently 1, 2 or 3. Such radicals include the polyether radicals —O—(CH₂)₁₋₃OH, —O—(CH₂)₁₋₃O(C₁-C₃alkyl)-O—(CH₂)₁₋₃—O—(CH₂)₁₋₃OH, and —O—(CH₂)₁₋₃—O—(CH₂)₁₋₃O(C₁-C₃alkyl) or
Q¹ and R^{A}, taken together with the nitrogen to which they are attached form an optionally substituted heterocyclic ring of 5 or 6 ring atoms, for example a cyclic amino group such as morpholino, piperidinyl, piperazinyl or methylpiperidinyl or a fluoro substituted cyclic amino group such as those of formulae (A)-(D):

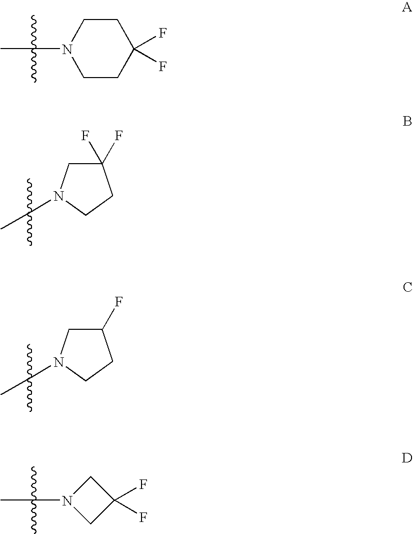

Case (e) arises when p is 0 or 1, q is 0, and r is 0 or 1. In this case, Q¹ is (i) hydrogen or an optional substituent which is not linked to -(Alk³)ₚ-(Z¹)_{q}-(Alk⁴)ᵣ- through a nitrogen atom or (ii) an optionally substituted carbocyclic radical; or (iii) an optionally substituted heterocyclic of 5 or 6 ring atoms which is not linked to -(Alk³)ₚ-(Z¹)_{q}-(Alk⁴)ᵣ- through a ring nitrogen; or (iv) a radical —CH₂[O(CH₂)_{w}]ₓZ² wherein Z² is H, —OH or —OCH₃ wherein x and w are independently 1, 2 or 3. In this case (e), Q¹ may be, for example:
a non-amino optional substituent, for example chloro, C₁-C₃alkoxy, cyano or acetyl; or a cyclopropyl, cylopentyl or cyclohexyl group;
a heterocyclic group such as any of those heteroaryl groups referred to above as examples of ring A, or a non aromatic heterocyclic group such as one having formula E defined above; or
a radical —CH₂[O(CH₂)ₓ]ₓZ² wherein Z² is H, —OH or —OCH₃ wherein x and w are independently 1, 2 or 3. Such radicals include the polyether radicals —O—(CH₂)₁₋₃OH, —O—(CH₂)₁₋₃O(C₁-C₃alkyl), —O—(CH₂)₁₋₃—O—(CH₂)₁₋₃OH, and —O—(CH₂)₁₋₃—O—(CH₂)₁₋₃O(C₁-C₃alkyl).

It will be apparent that compounds of the invention includes a subclass wherein the radical comprising ring A and substituents R₃, R₄ and S₁ is a radical of formula (IC),

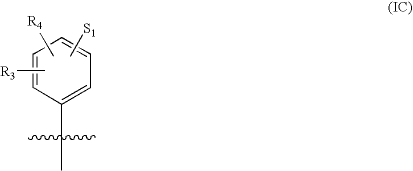

wherein R₃ and R₄ are as defined and discussed above, and S₁ is hydrogen, or a substituent selected from fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or S₁ and R₃, or S₁ and R₄, together represent methylenedioxy (—OCH₂O—) or ethylenedioxy ((—OCH₂ CH₂O—) in either of which one or more hydrogens are optionally replaced by fluorine; or S₁ is a radical of formula (IB):

-(Alk³)ₚ-(Z¹)_{q}-(Alk⁴)ᵣ-Q¹ (IB)

wherein
p, q and r are independently 0 or 1;
Z¹ is —O—, —S—, —(C═O)—, —(C═S)—, —S(O)—, —SO₂—, —NR^{A}—, or, in either orientation, —C(═O)N(R^{A})— or —SO₂NR^{A}—;
Q¹ is (i) hydrogen or an optional substituent; or (ii) an optionally substituted carbocyclic or heterocyclic radical; or (iii) a radical —CH₂[O(CH₂)_{w}]ₓZ² wherein
Z² is H, —OH or —O(C₁-C₃alkyl) wherein x and w are independently 1, 2 or 3;
R^{A} is hydrogen or C₁-C₃ alkyl optionally substituted with one or more fluorine substituents; and
Alk³ and Alk⁴ are divalent C₁-C₃ alkylene or C₂-C₃ alkenylene radicals, each optionally substituted by one or two substituents selected from fluoro, chloro, C₁-C₃alkyl optionally substituted with one or more fluorine substituents, C₁-C₃alkoxy optionally substituted with one or more fluorine substituents.

In the immediately foregoing subclass of compounds of the invention, it is presently preferred that S₁ be in the meta position of the ring.

Also in the foregoing subclass S₁ may be hydrogen, or a substituent selected from fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or S₁ and R₃, or S₁ and R₄, together may represent methylenedioxy (—OCH₂O—) or ethylenedioxy (—OCH₂CH₂O—) in either of which one or more hydrogens are optionally replaced by fluorine;

Alternatively in the foregoing subclass S₁ may be a radical of formula (IB):

-(Alk³)ₚ-(Z¹)_{q}-(Alk⁴)ᵣ-Q¹ (IB)

wherein
p, q and r are independently 0 or 1;
Z¹ is —O—, —S—, —(C═O)—, —(C═S)—, —S(O)—, —SO₂—, —NR^{A}—, or, in either orientation, —C(═O)N(R^{A})— or —SO₂NR^{A}—;
Q¹ is (i) hydrogen or an optional substituent; or (ii) an optionally substituted carbocyclic or heterocyclic radical; or (iii) a radical —CH₂[O(CH₂)_{w}]ₓZ² wherein
Z² is H, —OH or —O(C₁-C₃alkyl) wherein x and w are independently 1, 2 or 3;
R^{A} is hydrogen or C₁-C₃ alkyl optionally substituted with one or more fluorine substituents; and
Alk³ and Alk⁴ are divalent C₁-C₃ alkylene or C₂-C₃ alkenylene radicals, each optionally substituted by one or two substituents selected from fluoro, chloro, C₁-C₃alkyl optionally substituted with one or more fluorine substituents, C₁-C₃alkoxy optionally substituted with one or more fluorine substituents.

In the subclass of compounds just discussed, when S₁ is a radical of formula (IB), Z¹, Q¹, R^{A}, Alk³ and Alk⁴ therein may be any of those radicals or groups defined and discussed in relation to compounds (I), cases (a), (b), (c), (d) or (e) above.

Specific compounds with which the invention is concerned include those of the Examples, particularly those exemplified compounds which have structure (III) above.

There are multiple synthetic strategies for the synthesis of the compounds (I) with which the present invention is concerned, but all rely on known chemistry, known to the synthetic organic chemist. Thus, compounds according to formula (I) can be synthesised according to procedures described in the standard literature and are well-known to the one skilled in the art. Typical literature sources are “Advanced organic chemistry”, 4^{th} Edition (Wiley), J March, “Comprehensive Organic Transformation”, 2^{nd} Edition (Wiley), R. C. Larock, “Handbook of Heterocyclic Chemistry”, 2^{nd} Edition (Pergamon), A. R. Katritzky), review articles such as found in “Synthesis”, “Acc. Chem. Res.”, “Chem. Rev”, or primary literature sources identified by standard literature searches online or from secondary sources such as “Chemical Abstracts” or “Beilstein”. Such literature methods include those of the preparative Examples herein, and methods analogous thereto.

For example, aryl substituents may be incorporated selectively at the 6 position of the purine ring system by using a palladium catalysed cross coupling reaction with a tetrahydropyran protected 2,6 dichloro purine and a substituted aryl boronic acid, suitable solvents are (though not limited to) DMF/H₂O or 1,4 dioxane.

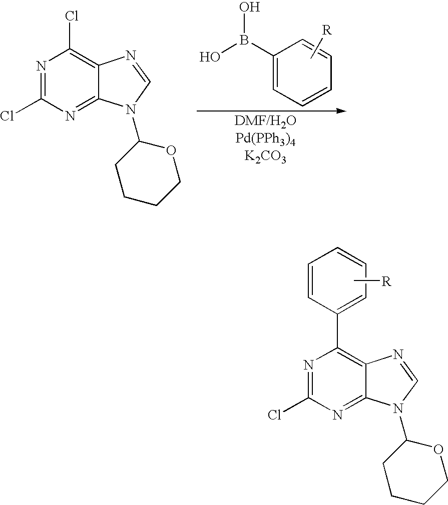

The compounds of the invention are inhibitors of HSP90 and are useful in the treatment of diseases which are responsive to inhibition of HSP90 activity such as cancers; viral diseases such as Hepatitis C(HCV) (Waxman, 2002); resensitisation of previously resistant fungal strains to the commonly used azole antifungal agents (e.g. fluconazole) as well as newer agents such as echinocandins (see Cowen and Lindquist, Science, Vol 309, 30 Sep. 2005, 2185-2189.); Immunosupression such as in transplantation (Bijlmakers, 2000 and Yorgin, 2000); Anti-inflammatory diseases (Bucci, 2000) such as Rheumatoid arthritis, Asthma, MS, Type I Diabetes, Lupus, Psoriasis and Inflammatory Bowel Disease; Cystic fibrosis (Fuller, 2000); Angiogenesis-related diseases (Hur, 2002 and Kurebayashi, 2001): diabetic retinopathy, haemangiomas, psoriasis, endometriosis and tumour angiogenesis. Also an Hsp90 inhibitor of the invention may protect normal cells against chemotherapy-induced toxicity and be useful in diseases where failure to undergo apoptosis is an underlying factor. Such an Hsp90 inhibitor may also be useful in diseases where the induction of a cell stress or heat shock protein response could be beneficial, for example, protection from hypoxia-ischemic injury due to elevation of Hsp70 in the heart (Hutter, 1996 and Trost, 1998) and brain (Plumier, 1997 and Rajder, 2000). An Hsp90 inhibitor—induced increase in Hsp70 levels could also be useful in diseases where protein misfolding or aggregation is a major causal factor, for example, neurogenerative disorders such as scrapie/CJD, Huntingdon's and Alzheimer's (Sittler, 2001; Trazelt, 1995 and Winklhofer, 2001)”.

Accordingly, the invention also includes:

(i) A pharmaceutical or veterinary composition comprising a compound of formula (I) above, together with a pharmaceutically or veterinarily acceptable carrier.
(ii) The use of a compound a compound of formula (I) above in the preparation of a composition for composition for inhibition of HSP90 activity in vitro or in vivo.
(iii). A method of treatment of diseases or conditions which are responsive to inhibition of HSP90 activity in mammals which method comprises administering to the mammal an amount of a compound of formula (I) above effective to inhibit said HSP90 activity.

It will be understood that the specific dose level for any particular patient will depend upon a variety of factors including the activity of the specific compound employed, the age, body weight, general health, sex, diet, time of administration, route of administration, rate of excretion, drug combination and the causative mechanism and severity of the particular disease undergoing therapy. In general, a suitable dose for orally administrable formulations will usually be in the range of 0.1 to 3000 mg, once, twice or three times per day, or the equivalent daily amount administered by infusion or other routes. However, optimum dose levels and frequency of dosing will be determined by clinical trials as is conventional in the art.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

### The Following Examples Illustrate the Preparation and Activities of Specific Compounds of the Invention:

### General Procedures

All reagents obtained from commercial sources were used without further purification. Anhydrous solvents were obtained from commercial sources and used without further drying. Flash chromatography was performed with pre-packed silica gel cartridges (Strata SI-1; 61 Å, Phenomenex, Cheshire UK or IST Flash II, 54 Å, Argonaut, Hengoed, UK). Thin layer chromatography was conducted with 5×10 cm plates coated with Merck Type 60 F₂₅₄ silica gel.

The compounds of the present invention were characterized by LC/MS using a Hewlett Packard 1100 series LC/MSD linked to quadripole detector (ionization mode: electron spray positive; column: Phenomenex Luna 3u C18(2) 30×4.6 mm; Buffer A prepared by dissolving 1.93 g ammonium acetate in 2.5 L HPLC grade H₂0 and adding 2 mL formic acid. Buffer B prepared by adding 132 mL buffer A to 2.5 L of HPLC grade acetonitrile and adding 2 mL formic acid; elution gradient 95:5 to 5:95 buffer A: buffer B over 3.75 minutes. Flow rate=2.0 mL/min)

Nuclear magnetic resonance (NMR) analysis was performed with a Brucker DPX-400 MHz NMR spectrometer. The spectral reference was the known chemical shift of the solvent. Proton NMR data is reported as follows: chemical shift (δ) in ppm, multiplicity (s=singlet, d=doublet, t=triplet, q=quartet, p=pentet, m=multiplet, dd=doublet of doublet, br=broad), coupling constant, integration.

Some compounds of the invention were purified by preparative HPLC. Preparative HPLC purifications were performed on a Waters FractionLynx MS Autopurification system with a Gemini® 5 μM C18(2), 100 mm×20 mm i.d. column from Phenomenex, running at a flow rate of 20 mL min⁻¹ with UV diode array detection (210-400 nm) and mass-directed collection. Gradients used for each compound are shown in Table 1.

At pH 4:Solvent A: HPLC grade Water+10 mM ammonium acetate+0.08% v/v formic acid.
Solvent B: 95% v/v HPLC grade acetonitrile+5% v/v Solvent A+0.08% v/v formic acid.
At pH 9:Solvent A: HPLC grade Water+10 mM ammonium acetate+0.08% v/v ammonia solution.
Solvent B: 95% v/v HPLC grade acetonitrile+5% v/v Solvent A+0.08% v/v ammonia solution.

The mass spectrometer was a Waters Micromass ZQ2000 spectrometer operating in positive or negative ion electrospray ionisation modes, with a molecular weight scan range of 150 to 1000

TABLE 1
Preparative HPLC gradients

| | % B for | % B for | % B for | % B for |
|---|---|---|---|---|
| Time/ | Compound no | Compound no | Compound no | Compound no |
| min | 8 | 9 | 11 | 12 |
| 0.0 | 5 | 5 | 5 | 5 |
| 0.5 | 20 | 25 | 30 | 35 |
| 7.0 | 40 | 45 | 50 | 55 |
| 7.5 | 95 | 95 | 95 | 95 |
| 9.5 | 95 | 95 | 95 | 95 |
| 10 | 5 | 5 | 5 | 5 |

IUPAC chemical names were generated using AutoNom Standard.

Some compounds of the invention can be made (by way of example) by following the route outlined in scheme 1. Experimental Methods, reagents and product isolation methods will be known to those skilled in the art of organic synthesis. It is understood that other methods can also be used.

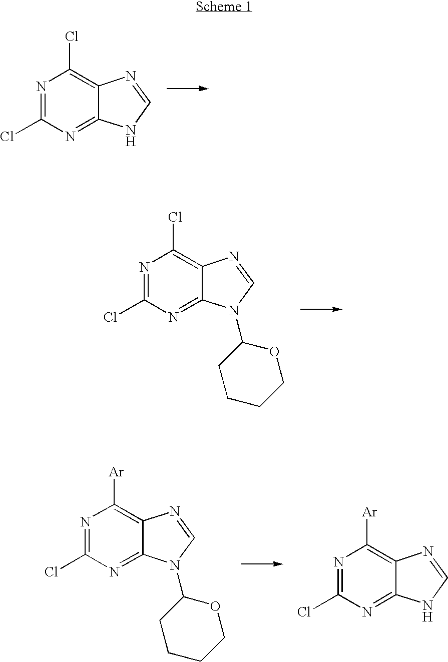

Some compounds of the invention can be made by following the route outlined in scheme 2.

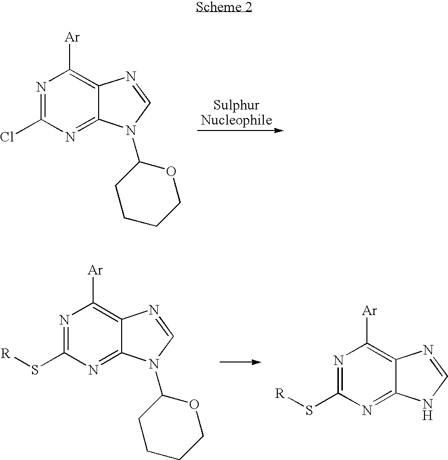

### Example 1

### 2-Chloro-6-(2,4-dichloro-phenyl)-9H-purine

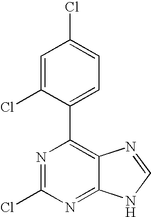

### Step 1

### 2,6-Dichloro-9-(tetrahydro-pyran-2-yl)-9H-purine

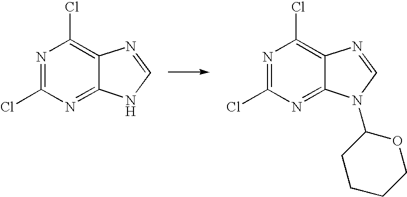

To 25 mL of dry ethyl acetate at 50° C. was added 2,6-dichloropurine (3 g, 16 mmols) followed by p-toluenesulfonic acid (43 mg, 0.226 mmols). The resulting solution was stirred vigorously and 3,4-dihydro-2H-pyran (1.63 mL, 17.9 mmols) was added drop-wise over 5-10 mins. This was stirred at 50-60° C. for one hour, then left to cool to ambient temperature. 2 mL ammonia hydroxide was added and the solution stirred for a further 5 minutes after which it was extracted with water (two times) and the ethyl acetate layer was dried over MgSO₄ and removed under vacuum to give a pale yellow solid. This crude product was either re-crystallized from hot ethyl acetate, or suspended in boiling hexane for 10 minutes then filtered. 3.191 g 73% yield

LC-MS retention time: 2.120 min; [M+H]⁺=191, 189 (run time 3.75 minutes)

### Step 2

### 2-chloro-6-(2,4-dichloro-phenyl)-9-(tetrahydro-pyran-2-yl)-9H-purine

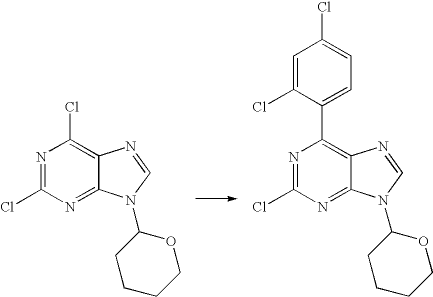

To (150 mg, 0.55 mmols) of 2,6-Dichloro-9-(tetrahydro-pyran-2-yl)-9H-purine in DMF (4 mL) was added 2,4-dichlorophenylboronic acid (115 mg, 0.605 mmols) and potassium carbonate (228 mg, 1.65 mmols). This was degassed by bubbling a stream of nitrogen through the solution over 5 minutes. Tetrakis (triphenylphosphine) palladium (0) (cat.) was added and mixture was degassed for a further minute, then heated to 100° C. under nitrogen for 14 hours. Reaction mixture was cooled to ambient temperature and saturated sodium chloride was added. Organics were extracted with ethyl acetate (three times), and the combined organic phases were, dried over MgSO₄ and concentrated under vacuum. This was then purified by flash column chromatography eluting DCM to 6% methanol/DCM, better conditions were found to be; hexane to 50% ethyl acetate/hexane.

LC-MS retention time: 2.398 minutes [M+H]⁺=383+385 and 299+301 (run time 3.75 minutes)

### Step 3

### 2-Chloro-6-(2,4-dichloro-phenyl)-9H-purine

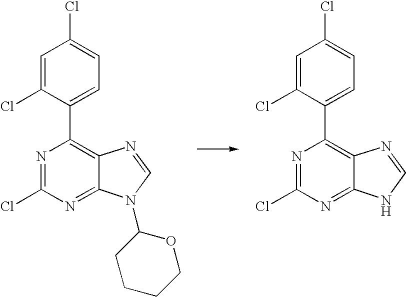

To 2-chloro-6-(2,4-dichloro-phenyl)-9-(tetrahydro-pyran-2-yl)-9H-purine in 2 mL 1,4-dioxane was added 2 mL 4M HCl in 1,4-dioxane. This was stirred for 15 minutes after which the solvents were evaporated and the residue purified by preparative HPLC at pH 4.

Alternatively this compound may be prepared applying a methanol solution of 2-chloro-6-(2,4-dichloro-phenyl)-9-(tetrahydro-pyran-2-yl)-9H-purine to an ion exchange column (IST SCX II, Argonaut, Hengoed, UK), eluting with methanol then with 7M ammonia in methanol to afford the de-protected product after removal of fraction solvents in vacuo.

LC-MS retention time minutes 1.889 [M+H]⁺=301+299 (run time 3.75 minutes) This compound has activity “A” in the fluorescence polarization assay described below.

### Example 2

### 6-(5-Benzyloxy-2,4-dichloro-phenyl)-2-chloro-9H-purine

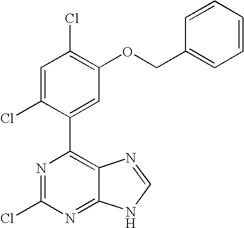

Step 1

### 1-Benzyloxy-2,4-dichloro-5-nitro-benzene

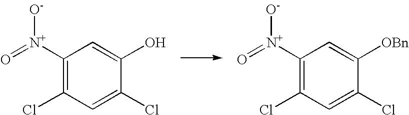

Potassium carbonate (12 g, 87 mmol) was added to a solution of 2,4-dichloro-5-nitrophenol (Lancaster Synthesis, Morecambe, Lancashire, UK) (15.6 g, 75 mmol) in acetone. Benzyl bromide (9 ml, 76 mmol) was added and the suspension heated at 75° C. (oil bath temperature) for ˜3 hrs. The resulting suspension was allowed to cool and water (500 ml) was added, the mixture was extracted with dichloromethane (2×200 ml). The combined extracts were washed with aqueous sodium hydroxide (150 ml, 2M), water (2×200 ml) and saturated aqueous sodium chloride solution (150 ml). The solution was dried over anhydrous sodium sulphate and concentrated to a pale yellow solid (21.5 g, 96%).

R_{f} 0.73 CH₂Cl₂ (SiO₂)

LC retention time 2.915 min; [M+H]⁺ no ionisation (run time 3.75 min)

### Step 2

### 5-Benzyloxy-2,4-dichloro-phenylamine

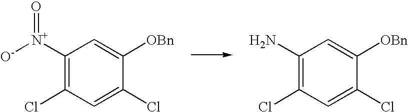

Iron powder (21 g, 376 mmol) was added to a suspension 1-Benzyloxy-2,4-dichloro-5-nitro-benzene (21.5 g, 72 mmol) in acetic acid (300 ml)/water (150 ml) and the mixture was heated at 85° C. (oil bath temperature) for ˜90 mins. The resulting suspension was filtered. The filtrate was allowed to cool, water (750 ml) was added and the mixture extracted with dichloromethane (3×150 ml). The combined extracts were washed with aqueous sodium hydroxide (300 ml, 2M), water (2×500 ml) and saturated aqueous sodium chloride solution (200 ml). The solution was dried over anhydrous sodium sulphate filtered and the filtrate solvents removed in vacuo to afford product as a pale brown solid (18.6 g, 96%)

R_{f} 0.57 CH₂Cl₂ (SiO₂)

LC retention time: 2.792 min; [M+H]⁺=270/268 (run time 3.75 min)

### Step 3

### 1-Benzyloxy-2,4-dichloro-5-iodo-benzene

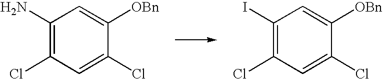

Hydrochloric acid (60 ml, 6M) was added to a solution of the 5-Benzyloxy-2,4-dichloro-phenylamine (16.2 g, 60 mmol) in acetic acid (240 ml) and the resulting suspension cooled (ice/water/salt). Aqueous sodium nitrite (4.8 g, 69.5 mmol in 40 ml) was added slowly (keeping the temperature <5° C.). On complete addition the resulting solution was stirred for ˜30 mins.

The resulting solution was poured into a solution of potassium iodide (20 g, 120 mmol) and iodine (4 g, 16 mmol) in water (200 ml), and the mixture stirred for ˜90 mins. Water (800 ml) was added and the mixture extracted with dichloromethane (3×250 ml). The combined extracts were washed with aqueous sodium thiosulphate solution (2×150 ml, 10%), aqueous sodium hydroxide (250 ml, 2M), water (2×250 ml) and saturated aqueous sodium chloride solution (200 ml). The solution was dried over anhydrous sodium sulphate and concentrated to a pale brown oil, solidified on standing. (20.6 g, 90%)

R_{f} 0.82 CH₂Cl₂ (SiO₂)

LC retention time: 3.084 min; [M+H]⁺ No ionisation (run time 3.75 min)

### Step 4

### 6-(5-Benzyloxy-2,4-dichloro-phenyl)-2-chloro-9H-purine

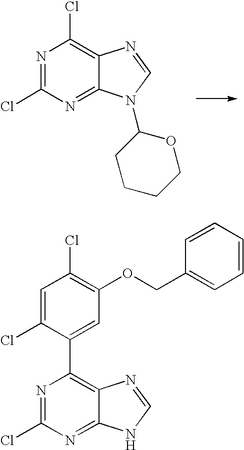

Potassium acetate (140 mg; 3 eq) was added to a solution of 1-Benzyloxy-2,4-dichloro-5-iodo-benzene (180 mg 1 equiv) and bis(pinacolato)diboron (132 mg, 1.1 equiv) in DMF (20 mL) under a nitrogen atmosphere. Palladium acetate (5 mole %) was added and the mixture heated, oil bath temperature 90° C., for ˜18 hrs. The resulting solution solvents were removed in vacuo, and the residue taken up in ethyl acetate the solution was washed with water (3×) and saturated aqueous sodium chloride solution (1×). The solution was dried over anhydrous sodium sulphate and concentrated to a pale brown gum.

The residue was taken up in 1,4-dioxan (20 mL) and 2,6-Dichloro-9-(tetrahydro-pyran-2-yl)-9H-purine (117 mg, 0.9 equiv) and aqueous potassium phosphate (2 ml, 2M) added, under a nitrogen atmosphere. Dichloro bis(triphenylphosphine) palladium(II) (cat.) was added and the mixture heated, oil bath temperature 100° C., for ˜3 hrs. The mixture was allowed to cool and ethyl acetate (50 ml) added. The mixture was washed with saturated aqueous sodium chloride solution (50 ml) and evaporated to a brown oil. This was dissolved in methanol applied to a to an ion exchange column (IST SCX II, Argonaut, Hengoed, UK), eluting with methanol then with 7M ammonia in methanol to afford the de-protected product after removal of fraction solvents in vacuo. Crude product was purified by preparative HPLC (ph4) to afford product as a solid.

LC-MS retention time: 2.279 min; [M+H]⁺=407+405 (run time 3.75 minutes)

This compound has activity “A” in the fluorescence polarization assay described below.

The compounds contained within the following table (table 2) were prepared by way of the methods of example 1 and the route outline in scheme 1. The activities of each example compound in the fluorescence polarization assay described below are reported in the column “HSP90 IC₅₀”

TABLE 2

| Example | | | Retention | HSP90 |
|---|---|---|---|---|
| No. | Structure | [M + H] | time (min) | IC₅₀ |
| 3 | 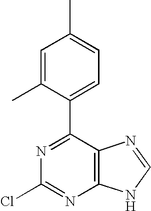 | 261, 259 | 1.828 | A |
| 4 | 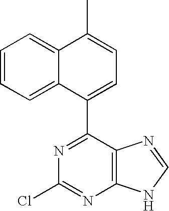 | 297, 295 | 1.935 | B |
| 5 | 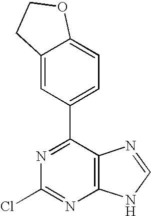 | 275, 273 | 1.843 | B |
| 6 | 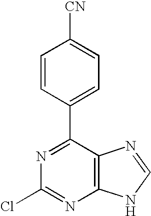 | 258, 256 | 1.838 | A |
| 7 | 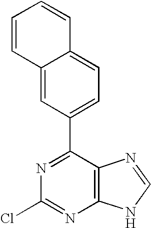 | 231 | 2.020 | B |
| 8 | 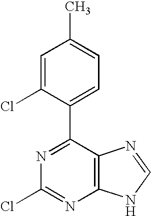 | 281, 279 | 2.025 | A |
| 9 | 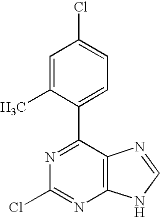 | 2.162 | 279, 281 | A |
| 10 | 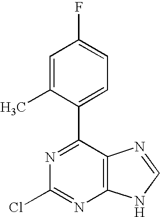 | 1.996 | 263 | A |
| 11 | 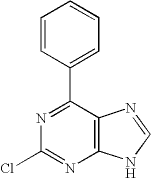 | 231 | 2.053 | A |

### Example 12

### 6-(5-benzyloxy-2,4-dichloro-phenyl)-2-methylsulfanyl-9H-purine

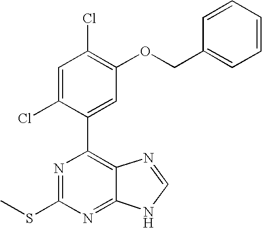

To 6-(5-Benzyloxy-2,4-dichloro-phenyl)-2-chloro-9-(tetrahydro-pyran-2-yl)-9H purine (step 4, example 2) in DMF (3 mL) was added sodium methanethiol (1.2 equiv), and the reaction mixture was heated to 120° C. for 10 minutes in a Smith microwave synthesizer. Saturated aqueous sodium bicarbonate solution was added to the reaction mixture (20 mL) and the organics were extracted ethyl acetate (2×25 mL), then washed with saturated sodium chloride solution solution (20 mL). The purine was deprotected by applying a methanol solution of the methanesulfanyl product to an ion exchange column (IST SCX II, Argonaut, Hengoed, UK), eluting with methanol then with 7M ammonia in methanol to afford the de-protected product after removal of fraction solvents in vacuo.

LC-MS retention time: 2.353 min; [M+H]⁺=419+417 (run time 3.75 minutes)

This compound has activity “A” in the fluorescence polarization assay described below

### Example 13

### 6-(4-cyano-phenyl)-2-methylsulfanyl-9H-purine

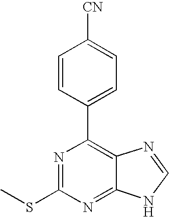

This compound was prepared by way of the method of example 12.

LC-MS retention time 1.939 min [M+H]⁺=268.1 (run time 3.75 minutes)

This compound has activity “A” in the fluorescence polarization assay described below

### Example 14

### 6-(2,4-dichloro-phenyl)-2-methylsulfanyl-9H-purine

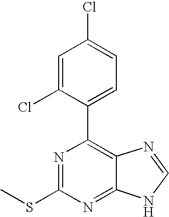

This compound was prepared by way of the method of example 12 from 2-chloro-6-(2,4-dichloro-phenyl)-9-(tetrahydro-pyran-2-yl)-9H-purine (example 1).

LC-MS retention time: 2.242 min; [M+H]⁺=313, 311 (run time 3.75 minutes)

This compound has activity “A” in the fluorescence polarization assay described below

### Example 15

### 6-(2-methyl-4-fluoro-phenyl)-2-methylsulfanyl-9H-purine

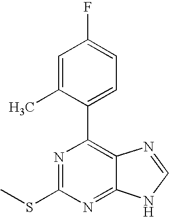

This compound was prepared by way of the method of example 12, and the routes outlined in scheme 1 and scheme 2.

LC-MS retention time 2.109 min; [M+H]⁺=275 (run time 3.75 minutes).

This compound has activity “A” in the fluorescence polarization assay described below.

The compounds contained within the following table (table 3) were prepared by way of the methods of example 1 and example 2 and the route outline in scheme 1 and scheme 2. Appropriate boronic acids and sulphur based nucleophiles were used for each example and will be known to those skilled in the art. The activities of each example compound in the fluorescence polarization assay described below are reported in the column “HSP90 IC₅₀”

TABLE 3

| Example | | | Retention | HSP90 |
|---|---|---|---|---|
| No. | Structure | [M + H] | time (min) | IC₅₀ |
| 16 | 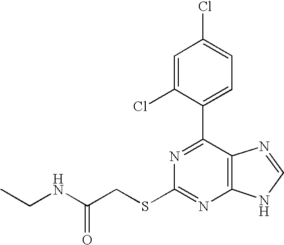 | 384, 382 | 1.971 | A |
| 17 | 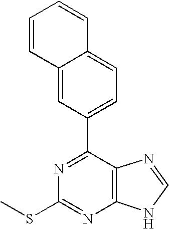 | 293 | 2.482 | B |
| 18 | 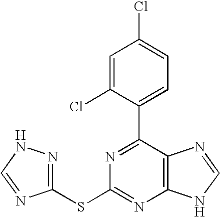 | 366,364 | 1.857 | A |
| 19 | 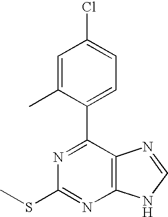 | 293, 291 | 2.291 | A |
| 20 | 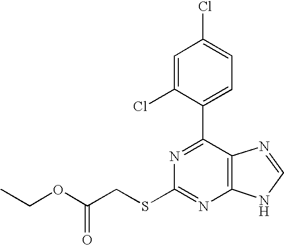 | 285, 383 | 2.294 | A |
| 21 | 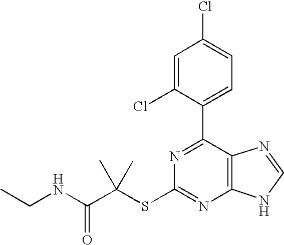 | 410, 412 | 2.183 | B |
| 22 | 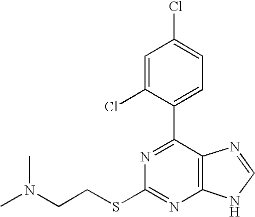 | 370, 370 | 1.594 | A |

### Example 23

### 6-(2,4-Dichloro-phenyl)-2-methoxy-9H-purine

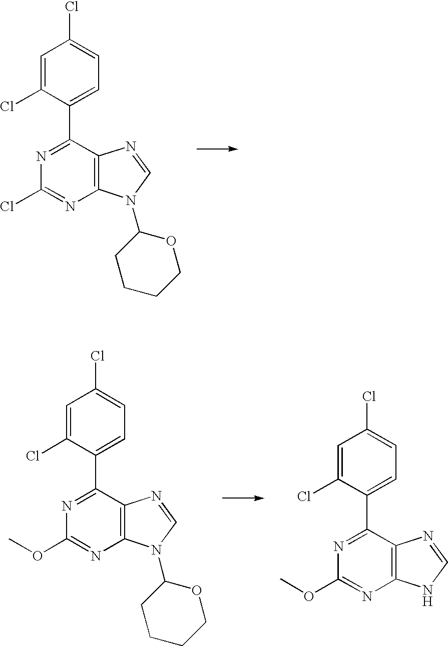

2-chloro-6-(2,4-dichloro-phenyl)-9-(tetrahydro-pyran-2-yl)-9H-purine (example 1; step 2) (0.070 g; 0.183 mmol) was dissolved in DMF (2 ml) and sodium methoxide was added (0.013 g; 0.237 mmol). The reaction mixture was sealed in a vial and heated in a microwave synthesiser at 125° C. for 1 hour. Reaction mixture was poured into sat. aqueous sodium bicarbonate solution and extracted with ethyl acetate. The organic phase was washed with sat NaCl solution and dried over sodium sulphate. Mixture was filtered and filtrate solvents removed in vacuo. Crude product was purified by flash chromatography on SiO₂, (0 to 10% ethyl acetate in hexane). THP protecting group was removed by applying a methanol solution of 2-methoxy-6-(2,4-dichloro-phenyl)-9-(tetrahydro-pyran-2-yl)-9H-purine to an ion exchange column (IST SCX II, Argonaut, Hengoed, UK), eluting with methanol then with 7M ammonia in methanol to afford the de-protected product after removal of fraction solvents in vacuo.

LC-MS retention time 2.041 min; [M+H]⁺=297, 295 (run time 3.75 minutes).

This compound has activity “A” in the fluorescence polarization assay described below.

### Example 24

### {4-[2,4-Dichloro-5-(2-chloro-9H-purin-6-yl)-phenoxymethyl]-benzyl}-diethyl-amine

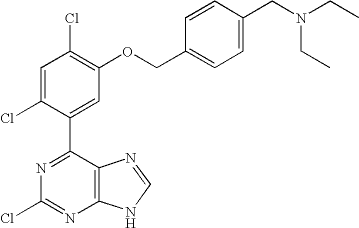

### Step 1

### 4-(2,4-Dichloro-5-nitro-phenoxymethyl)-benzoic Acid Methyl Ester

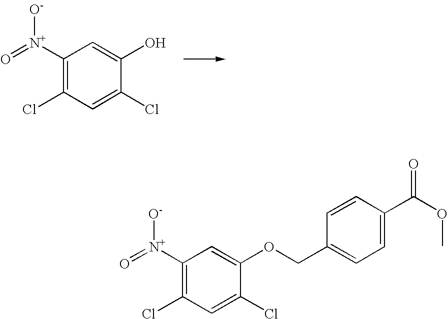

Potassium carbonate was added to a solution of the 2,4-dichloro-5-nitrophenol in acetone. Methyl (4-brommethyl)benzoate was added and the suspension heated, 75° C., for ˜3 hrs. The resulting suspension was allowed to cool and water added, the mixture was extracted with dichloromethane. The combined extracts were washed with aqueous sodium hydroxide, water and saturated aqueous sodium chloride solution. The solution was dried over anhydrous sodium sulphate and concentrated to a pale yellow solid.

### Step 2

### 4-(5-Amino-2,4-dichloro-phenoxymethyl)-benzoic acid methyl ester

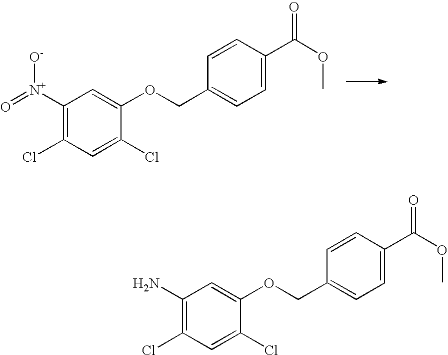

Title compound was made by way of the methods of example 2 step 2.

### Step 3

### 4-(2,4-Dichloro-5-iodo-phenoxymethyl)-benzoic acid methyl ester

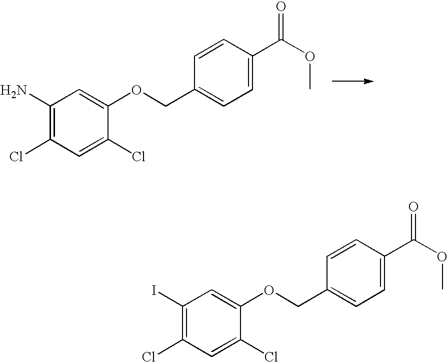

Title compound was made by way of the methods of example 2 step 3.

### Step 4

### [4-(2,4-Dichloro-5-iodo-phenoxymethyl)-phenyl]-methanol

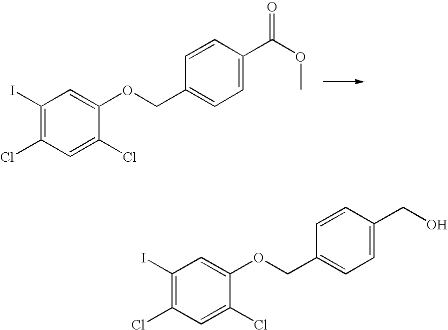

Diisobutylaluminium hydride solution (1M in dichloromethane) was added to a solution of 4-(2,4-Dichloro-5-iodo-phenoxymethyl)-benzoic acid methyl ester in dichloromethane at −78° C., under a nitrogen atmosphere. The solution was stirred at −78° C., for ˜60 mins and at room temperature for ˜2 hrs. The resulting solution was cooled −78° C. and methanol added. The solution was stirred at room temperature for ˜60 mins. Dichloromethane was added and the solution washed with water and saturated aqueous sodium chloride solution. The solution was dried over anhydrous sodium sulphate and concentrated to a brown solid. The crude product was purified by column chromatography, silica, eluting with dichloromethane to give the product as an off-white solid.

### Step 5

### 4-(2,4-Dichloro-5-iodo-phenoxymethyl)-benzaldehyde

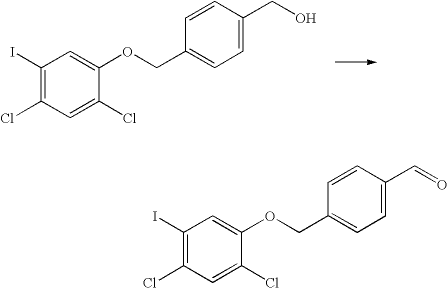

Manganese dioxide was added to a solution of [4-(2,4-Dichloro-5-iodo-phenoxymethyl)-phenyl]-methanol in ethylene glycol dimethyl ether and the suspension stirred for ˜18 hrs. The resulting suspension was filtered and the filtrate concentrated to give the product as an off-white solid.

### Step 6

### {4-[2,4-Dichloro-5-(2-chloro-9H-purin-6-yl)-phenoxymethyl]-benzyl}-diethyl-amine

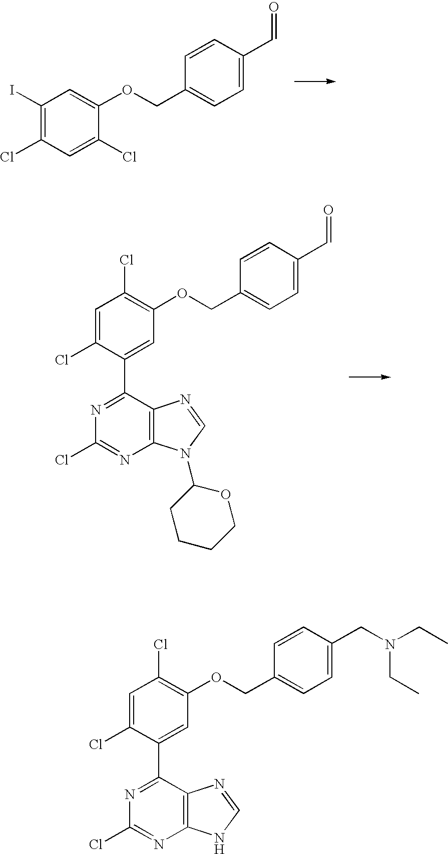

Potassium acetate was added to a solution of the 4-(2,4-Dichloro-5-iodo-phenoxymethyl)-benzaldehyde and bis(pinacolato)diboron in DMF under a nitrogen atmosphere. Palladium (II) acetate was added and the mixture heated, 90° C., for ˜18 hrs. The resulting solution was concentrated, and the residue taken up in ethyl acetate, the solution was washed with water and saturated aqueous sodium chloride solution. The solution was dried over anhydrous sodium sulphate and concentrated to a pale brown gum.

The residue was taken up in 1,4-dioxan and 2,6-Dichloro-9-(tetrahydro-pyran-2-yl)-9H-purine and aqueous potassium phosphate were added, under a nitrogen atmosphere. Dichloro bis(triphenylphosphine) palladium(II) (cat.) was added and the mixture heated, 100° C., for ˜3 hrs. The mixture was allowed to cool and ethyl acetate added. The mixture was washed with saturated aqueous sodium chloride solution. The solution was dried over anhydrous sodium sulphate and concentrated to an orange/brown gum. The crude product was purified by column chromatography, silica, eluting with mixtures of ethyl acetate and hexane to give the product as an off-white solid.

Diethylamine was added to a solution of 4-{2,4-Dichloro-5-[2-chloro-9-(tetrahydro-pyran-2-yl)-9H-purin-6-yl]-phenoxymeyhyl}-benzaldehyde in dichloromethane and the solution stirred. Sodium triacetoxyborohydride was added and the solution stirred for ˜72 hrs. Dichloromethane was added and the solution washed with saturated aqueous sodium hydrogen carbonate solution, water and saturated aqueous sodium chloride solution. Solution was dried over anhydrous sodium sulphate and concentrated to colourless gum. The crude product was purified by column chromatography, cation exchange resin, eluting with mixtures of dichloromethane and methanol and with mixtures of methanol and diisopropylethyl amine. The crude product was purified by preparative HPLC, to give the product as an off-white solid.

LC retention time 1.816 min; [M+H]⁺=490.05/492.1 (Run time 3.75 mins).

This compound has activity “A” in the fluorescence polarization assay described below.

### Example 25

### 6-(2,4-Dichloro-phenyl)-2-methanesulfonyl-7H-purine

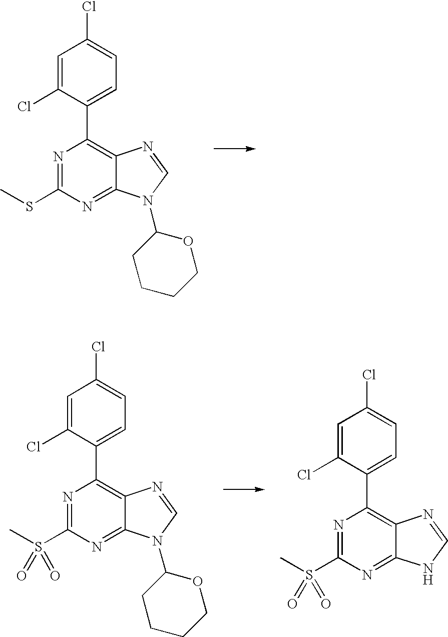

6-(2,4-Dichlorophenyl)-2-methylsulfanyl-9-(tetrahydropyran-2-yl)-9H-purine (example 1, step 2) (0.14 mmol) was dissolved in dichloromethane and cooled to 0° C. To this was added meta-chloroperoxybenzoic acid (0.29 mmol) portion wise, this was then stirred for 1.5 hours after which the reaction was quenched with 4 mL saturated sodium bicarbonate solution, the organics extracted ×2 ethyl acetate, washed saturated brine solution and dried MgSO₄. This was purified by flash column chromatography eluting hexane to 1:1 ethyl acetate/hexane. Product was de-protected using an SCX-II tosic acid column (Phenomenex) washing with methanol and releasing the deprotected product with 7N methanolic ammonia. This was further purified by preparative HPLC at pH 4.

LC-MS retention time: 1.964 min; [M+H]⁺=343+345 (run time 3.75 minutes)

This compound has activity “B” in the fluorescence polarization assay described below.

### Example 26

### 6-(2,4-Dichloro-phenyl)-2-methanesulfinyl-7H-purine

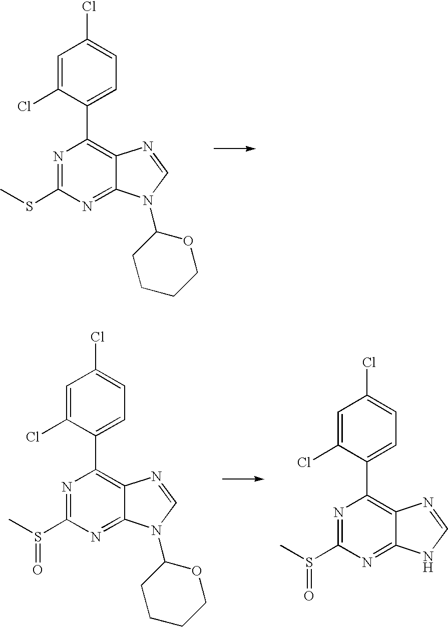

6-(2,4-Dichlorophenyl)-2-methylsulfanyl-9-(tetrahydropyran-2-yl)-9H-purine (0.3 mmol) (example 1, step 2) was dissolved in dichloromethane and cooled to 0° C. To this was added meta-chloroperoxybenzoic acid (0.24 mmol). This was stirred for 10 minutes, then quenched with 4 mL saturated sodium bicarbonate solution the organics extracted ×2 ethyl acetate, washed saturated brine solution and dried MgSO₄. Product was deprotected using an SCX-II tosic acid column washing with methanol and releasing the deprotected product with 7N methanolic ammonia. This was further purified by preparative HPLC at pH 4.

LC-MS retention time 1.808 min; [M+H]⁺=327+329 (run time 3.75 minutes)

This compound has activity “B” in the fluorescence polarization assay described below.

### Example 27

### 4-Chloro-5-(2-chloro-9H-puriny-6-yl)-2-methyl-phenol

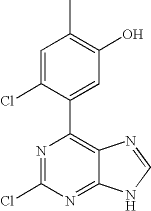

### Step 1

### Carbonic Acid 4-chloro-2-methyl-phenyl Ester Ethyl Ester

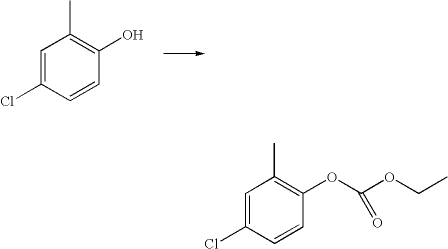

Pyridine (4.22 ml) was added to a solution of 4-chloro-2-methylphenol (5.0 g) in dichloromethane (35 ml). The mixture was cooled to 0° C. and ethyl chloroformate (3.69 ml) was added drop-wise. Reaction mixture was allowed to warm to ambient temperature and stir for 3 hours (a white ppt had formed). 2N HCl was added (25 ml) and the organic phases separated. Organic phase was washed with aq copper sulphate solution, then sat NaCl solution and dried over MgSO4.

Mixture was filtered and filtrate solvents removed in vacuo to afford a clear colourless oil. (7.19 g; 96%).

R_{f}=0.47 (1:9 EtOAc:Hexane).

### Step 2

### Carbonic Acid 4-chloro-2-methyl-5-nitro-phenyl Ester Ethyl Ester

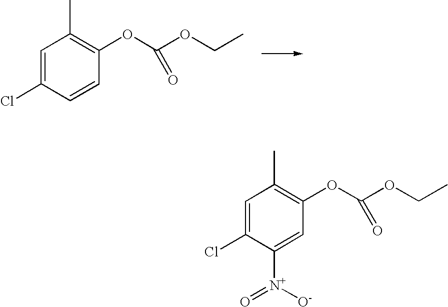

Conc. Nitric acid (11 ml) was added cautiously to ice bath cooled conc. Sulphuric acid and the mixture stirred at 0° C. Carbonic acid 4-chloro-2-methyl-phenyl ester ethyl ester was added drop-wise giving a yellow solution. Cooling bath was removed and mixture stirred for 4 hours, then poured slowly onto ice-water mix. The mixture was extracted with DCM (3×20 ml) and combined organics washed with 2N NaOH solution, then sat. NaCl (aq) solution and dried over MgSO₄. Mixture was filtered and filtrate solvents removed in vacuo to afford product as a yellow oil, (7.23 g, 84%).

### Step 3

### Carbonic Acid 5-Amino 4-chloro-2-methyl-phenyl Ester Ethyl Ester

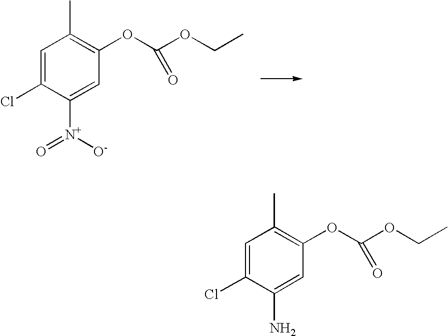

Iron powder (7.8 g) was added to a suspension of carbonic acid 4-chloro-2-methyl-5-nitro-phenyl ester ethyl ester (7.23 g) in Acetic acid (75 ml) and water (37 ml). Reaction mixture was heated to 85° C. for 90 mins. The hot suspension was filtered through a pad of celite and filtrate allowed to cool. Water was added and the mix was extracted with dichloromethane. The organic phase was washed with 2N NaOH solution (aq) then sat NaCl (aq) solution and dried over MgSO₄. Mixture was filtered and filtrate solvents removed in vacuo to afford product as a brown oil, (5.448 g, 85%).

### Step 4

### Carbonic Acid 4-chloro-2-methyl-5-iodo-phenyl Ester Ethyl Ester

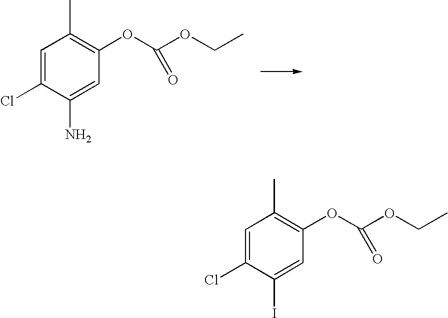

Carbonic acid 5-Amino 4-chloro-2-methyl-phenyl ester ethyl ester (5.48 g) was dissolved in acetic acid (45 m) and the mix cooled to 0° C. 6N HCl (15 ml) was added giving a suspension. To this suspension was added sodium Nitrite solution (1.96 g in 16.6 ml water) drop-wise such that internal temp remained less than 5° C. When addition was complete mixture was stirred at 0° C. for 30 minutes and then poured into and aqueous solution of potassium iodide (5.5 g) and iodine (1.8 g). Mixture was stirred for 90 mins at ambient temperature. Water (40 ml) was added and mix extracted with dichloromethane (3 times). Combined organics were washed with sodium thiosulphate solution (10% w/v), then sat. NaCl (aq) solution and dried over MgSO₄. Mixture was filtered and filtrate solvents removed in vacuo to afford product as a brown solid. (6.67 g, 83%).

### Step 5

### Carbonic Acid-4-chloro-2-methyl-5-(4,4,5,5-tetramethyl-[1,3,2]dioxaborolan-2-yl-phenyl Ester Ethyl Ester

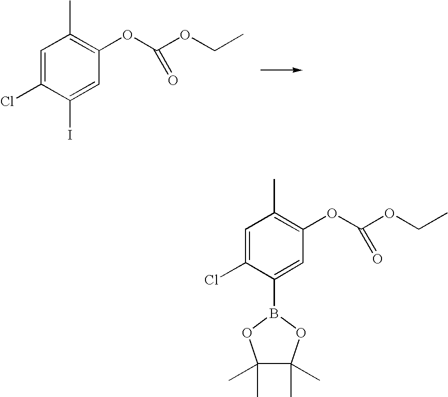

Carbonic acid 4-chloro-2-methyl-5-iodo-phenyl ester ethyl ester (6.67 g) was dissolved in DMF (40 ml) and bis-(pinacolato)diboron (5.23 g) was added followed by potassium acetate (5.77 g). This mixture was degassed by bubbling nitrogen gas through the mix for 10 mins. Palladium (II) acetate was added and mix was heated under nitrogen atmosphere to 90° C. for 18 hours. The reaction mixture was then allowed to cool, diluted with ethyl acetate, filtered theough pad of celite and organic filtrates washed with water (two times) and dried over MgSO₄. Mixture was filtered and filtrate solvents removed in vacuo to afford product as a brown solid.

### Step 6

### 4-Chloro-5-[2-chloro-9-(tetrahydro-pyran-2-yl)-9H-puriny-6-yl]-2-methyl-phenol

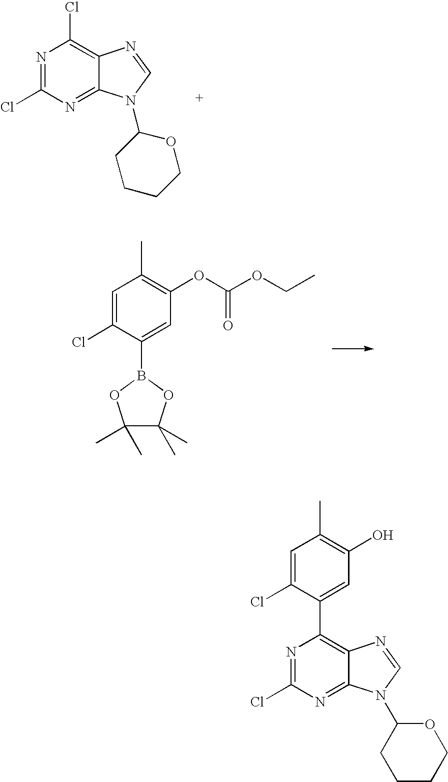

Carbonic acid-4-chloro-2-methyl-5-(4,4,5,5-tetramethyl-[1,3,2]dioxaborolan-2-yl-phenyl ester ethyl ester was dissolved in dioxane (20 ml) and K₃PO₄ (1.14 g; in 5.4 ml water) was added. This mixture was degassed by bubbling nitrogen gas through the mix for 10 mins. Dichlorobis(triphenyl-phosphine) palladium(II) (40 mg) was added and mix was heated under nitrogen atmosphere to 100° C. for 7 hours. 1,1′-bis(di-tert-butyl)ferrocene palladium (II) dichloride (cat amount) was added and heating continued over 24 hours. Reaction mixture was allowed to cool to ambient temperature and then diluted with ethyl acetate. Organic phase was washed with sat. NaCl (aq) solution and dried over MgSO₄. Mixture was filtered and filtrate solvents removed in vacuo to afford product as an oil. Crude product was purified by flash chromatography on silica gel eluting with 0 to 60% ethyl acetate in hexane (gradient) to afford title compound as an orange oil.

### Step 7

### 4-Chloro-5-(2-chloro-9H-puriny-6-yl)-2-methyl-phenol

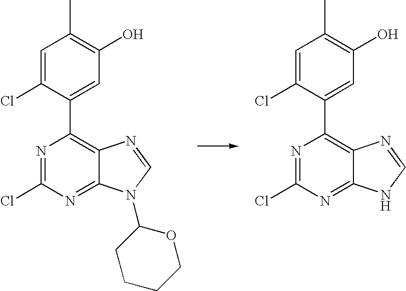

4-Chloro-5-[2-chloro-9-(tetrahydro-pyran-2-yl)-9H-puriny-6-yl]-2-methyl-phenol was de-protected using the SCX II methodology outlined in example 1 step 3.

LC-MS retention time 1.919 min; [M+H]⁺=297, 295 (run time 3.75 minutes)

This compound has activity “A” in the fluorescence polarization assay described below.

### Example 28

### {2-[4-Chloro-5-(2-chloro-9H-purin-6-yl)-2-methyl-phenoxy]-ethyl}-diethyl-amine

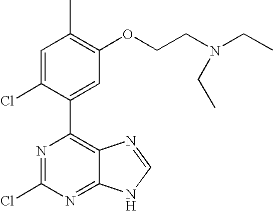

### Step 1

### (2-{4-Chloro-5-[2-chloro-9-(tetrahydro-pyran-2-yl)-9H-purin-6-yl]-2-methyl-phenoxy}-ethyl)-diethyl-amine

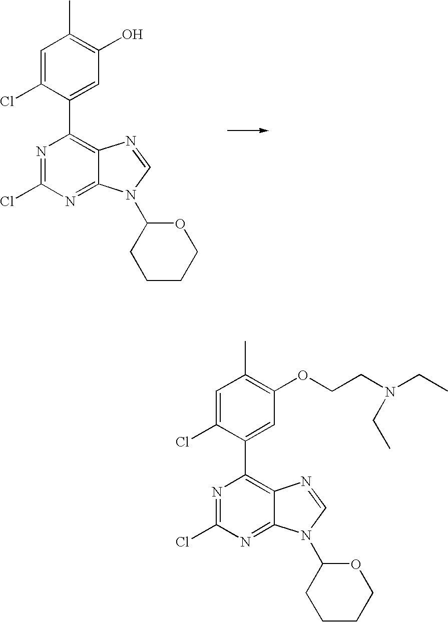

4-Chloro-5-[2-chloro-9-(tetrahydro-pyran-2-yl)-9H-puriny-6-yl]-2-methyl-phenol (200 mg) was dissolved in THF (10 ml) under a nitrogen atmosphere and triphenylphosphine (208 mg) was added followed by 2-(diethylamino)ethanol (0.084 ml). Diisopropylazodicarboxylate (0.155 ml) was added and reaction mixture was stirred at ambient temperature for 3 hours. Water was added and the mixture was extracted with ethyl acetate (three times) and the combined organics were washed with sat. aqueous sodium bicarbonate solution, then was washed with sat. NaCl (aq) solution and dried over MgSO₄. Mixture was filtered and filtrate solvents removed in vacuo to afford product as an oil. Crude product was purified by flash chromatography on silica gel eluting with 0 to 8% methanol in dichloromethane (gradient) to afford title compound as oil.

### Step 2

### {2-[4-Chloro-5-(2-chloro-9H-purin-6-yl)-2-methyl-phenoxy]-ethyl}-diethyl-amine

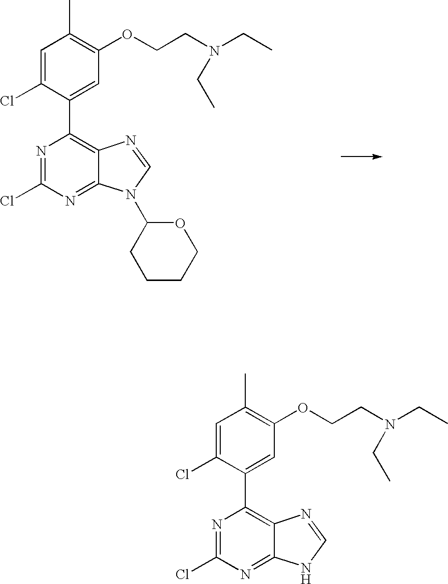

(2-{4-Chloro-5-[2-chloro-9-(tetrahydro-pyran-2-yl)-9H-purin-6-yl]-2-methyl-phenoxy}-ethyl)-diethyl-amine then dissolved in dioxane (2 ml) and 2 ml 4M HCl (aq) was added. Mixture was stirred for 2 hours and solvents removed in vacuo. The crude product was purified by prep HPLC (pH 4) to afford product as formate salt.

LC-MS retention time 1.605 Min, [M+H]⁺=396, 394 (run time 3.75 minutes)

This compound has activity “A” in the fluorescence polarization assay described below.

### Example 29

### {2-[4-Chloro-2-methyl-5-(2-methylsulfanyl-9H-purin-6-yl)-phenoxy]-ethyl}-diethyl-amine

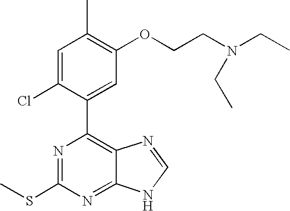

This compound was made by the method of example 12 from (2-{4-Chloro-5-[2-chloro-9-(tetrahydro-pyran-2-yl)-9H-purin-6-yl]-2-methyl-phenoxy}-ethyl)-diethyl-amine (example 28 step 1). Product was purified by prep HPLC (pH 4).

LC-MS retention time 1.694 min; [M+H]⁺=406 (run time 3.75 minutes)

This compound has activity “A” in the fluorescence polarization assay described below

### Example 30

### 2-{6-[2-Chloro-5-(2-diethlamino-ethoxy)-4-methyl-phenyl]-9H-purin-2-ylsulfanyl}-N-ethyl Acetamide

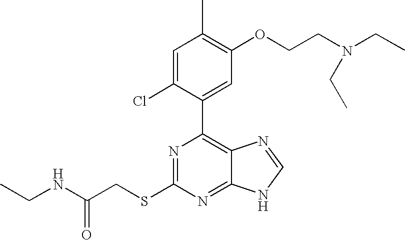

This compound was made by the methods of example 12 from (2-{4-Chloro-5-[2-chloro-9-(tetrahydro-pyran-2-yl)-9H-purin-6-yl]-2-methyl-phenoxy}-ethyl)-diethyl-amine (example 28 step 1) and N-ethyl-2-merapto-acetamide. Product was purified by prep HPLC (pH 4) to afford title compound as formate salt.

LC-MS retention time 1.584 min; [M+H]⁺=479 (run time 3.75 minutes)

This compound has activity “A” in the fluorescence polarization assay described below

### Example 31

### 8-Chloro-6-(2,4-dimethyl-phenyl)-2-methylsulfanyl-9H-purine

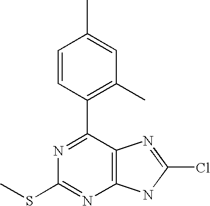

### Step 1

### 2-Chloro-6-(2,4-dimethyl-phenyl)-9-(tetrahydro-pyran-2-yl)-9H-purine

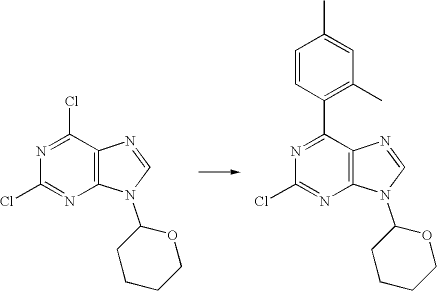

This compound was prepared by way of the method of example 1 from 2,4-dimethylphenylboronic acid and 2,6-dichloro-9-(tetrahydro-pyran-2-yl)-9H-purine (example 1, step 1).

LC-MS retention time: 2.65 min; [M+H]⁺=343 (run time 3.75 minutes)

### Step 2

### 6-(2,4-Dimethyl-phenyl)-2-methylsulfanyl-9-(tetrahydro-pyran-2-yl)-9H-purine

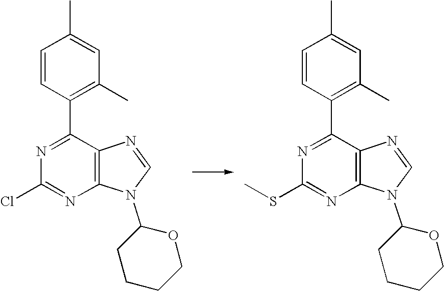

This compound was prepared by way of the method of example 12 from sodium methanethiolate and 2-Chloro-6-(2,4-dimethyl-phenyl)-9-(tetrahydro-pyran-2-yl)-9H-purine.

LC-MS retention time: 2.71 min; [M+H]⁺=355 (run time 3.75 minutes)

### Step 3

### 8-Chloro-6-(2,4-dimethyl-phenyl)-2-methylsulfanyl-9-(tetrahydro-pyran-2-yl)-9H-purine

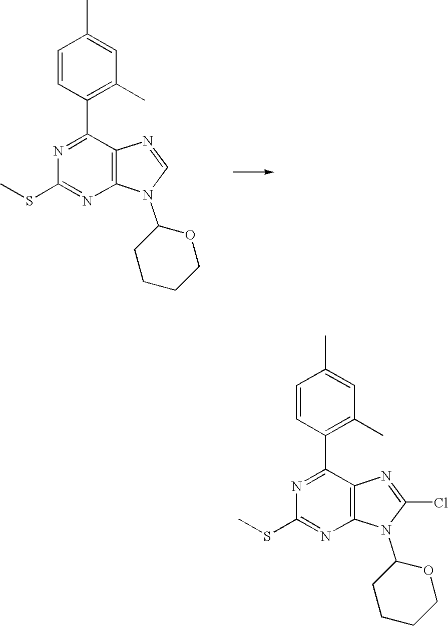

To a stirred solution of butyl lithium in hexanes (170 μL, 0.423 mmol, 2.5 M) at 0° C. was added diisopropylamine (59 μL, 0.423 mmol) in THF (0.5 mL). The mixture was stirred at 0° C. for 10 minutes then cooled to −78° C. A solution of 6-(2,4-dimethyl-phenyl)-2-methylsulfanyl-9-(tetrahydro-pyran-2-yl)-9H-purine) (50.0 mg, 0.141 mmol) in THF (0.5 mL) was added. The solution was stirred for 15 minutes then a solution of N-chlorosuccinimide (57.0 mg, 0.423 mmol) in THF (0.5 mL) was added dropwise. The mixture was stirred for 20 minutes then saturated aqueous sodium bicarbonate (5 mL) was added and the mixture allowed to warm to room temperature. The mixture was diluted with ethyl acetate then extracted with saturated aqueous sodium bicarbonate solution then washed with saturated brine. The organic extracts were dried over sodium sulphate, filtered and then concentrated in vacuo to give the crude product which was purified by flash column chromatography (eluting with ethyl acetate/hexane 2:5) to give the title compound as a white solid (21.1 mg)

LC-MS retention time minutes 2.88 [M-THP+H]⁺=305 (run time 3.75 minutes)

### Step 4

### 8-Chloro-6-(2,4-dimethyl-phenyl)-2-methylsulfanyl-9H-purine

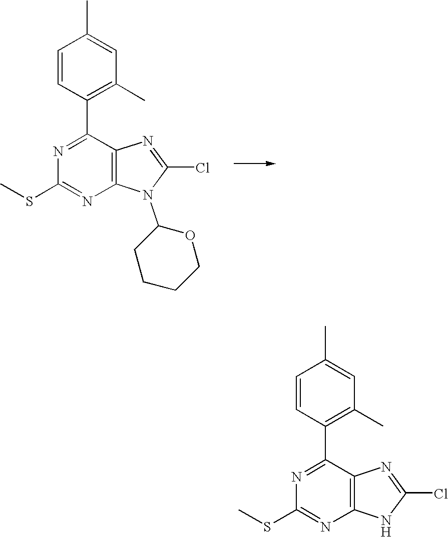

A solution of 8-Chloro-6-(2,4-dimethyl-phenyl)-2-methylsulfanyl-9-(tetrahydro-pyran-2-yl)-9H-purine (10.0 mg, 25.8 μmol) in methanol (1.0 mL) was loaded onto an ion exchange column (IST SCX II, Argonaut, Hengoed, UK). The column was then flushed with methanol and then the title compound was eluted using a solution of ammonia in methanol (7 M). The crude product was adsorbed onto silica and purified by flash column chromatography (eluting with hexane/ethyl 4:1 acetate) to give the title compound as a white solid (5.0 mg).

LC-MS retention time: 2.48 min; [M+H]⁺=305 (run time 3.75 minutes)

This compound has activity “B” in the fluorescence polarization assay described below.

### Example 32

### 6-(2,4-Dimethyl-phenyl)-8-methyl-2-methylsulfanyl-9H-purine

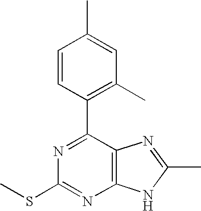

### Step 1

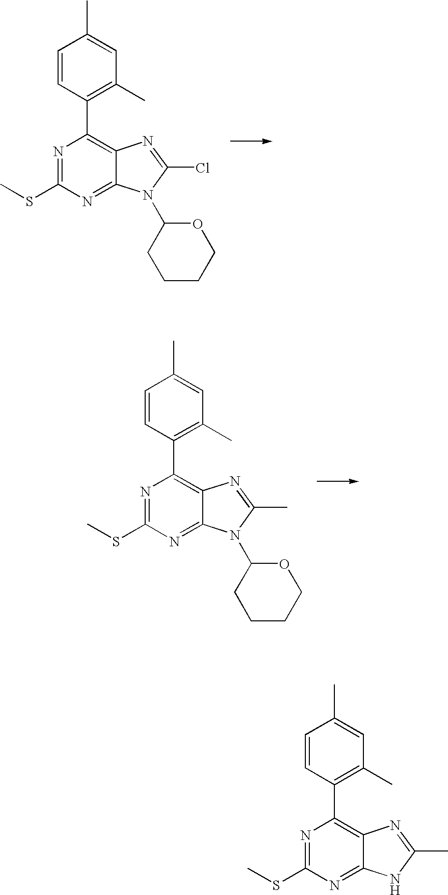

To a stirred solution of 8-Chloro-6-(2,4-dimethyl-phenyl)-2-methylsulfanyl-9-(tetrahydro-pyran-2-yl)-9H-purine (example 31 step 3) (25.0 mg, 67.9 μmol) in THF (2 mL) at −78° C. was added methyl lithium solution (100 μL, 170 μmol, 1.6M in diethyl ether) and the reaction stirred for 30 minutes at −78° C. then at 0° C. for 30 minutes. The reaction mixture was diluted with saturated sodium bicarbonate solution and then extracted with ethyl acetate. The organic extracts were then washed with saturated brine, dried over sodium sulphate then concentrated to a crude solid. This solid was then purified by flash column chromatography (eluting with ethyl acetate/hexane 1:1) then dissolved in methanol, loaded onto an ion exchange column (IST SCX II, Argonaut, Hengoed, UK). The column was then flushed with methanol and then the title compound was eluted using a solution of ammonia in methanol (7 M). Concentration in vacuo gave the title compound as a white solid (5.0 mg).

LC-MS retention time: 2.21 min; [M+H]⁺=285 (run time 3.75 minutes).

This compound has activity “B” in the fluorescence polarization assay described below.

### Example 33

### 6-(2,4-Dimethyl-phenyl)-2-methylsulfanyl-9H-purine-8-carbonitrile

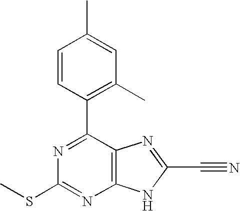

To a stirred solution of butyl lithium in hexanes (170 μL, 0.423 mmol, 2.5 M) at 0° C. was added diisopropylamine (59 μL, 0.423 mmol) in THF (0.5 mL). The mixture was stirred at 0° C. for 10 minutes then cooled to at −78° C. A solution of 6-(2,4-dimethyl-phenyl)-2-methylsulfanyl-9-(tetrahydro-pyran-2-yl)-9H-purine) (50.0 mg, 0.141 mmol) in THF (1.0 mL) was added. The solution was stirred for 25 minutes then a solution of p-toluenesulfonyl cyanide (77.0 mg, 0.423 mmol) in THF (1.0 mL) was added dropwise. The mixture was stirred for 1 hour then at 0° C. for 1 hour. The reaction mixture was partitioned between saturated aqueous sodium bicarbonate solution and ethyl acetate. The organic extract was then washed with saturated brine and then dried over sodium sulphate, filtered and then concentrated in vacuo to give a crude oil which was purified by flash column chromatography (eluting with ethyl acetate/hexane 1:8). The resultant intermediate was deprotected using an ion exchange column (as in example 31 step 4) to give the title compound as a white solid (9.0 mg)

LC-MS retention time: 2.60 min; [M+H]⁺=296 (run time 3.75 minutes).

This compound has activity “B” in the fluorescence polarization assay described below.

### Example 34

### {3-[2,4-Dichloro-5-(2-chloro-9H-purin-6-yl)-phenoxy]-propyl}-dimethyl-amine

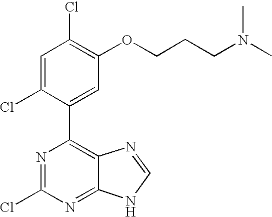

### Step 1

### 2,6-Dichloro-9-(4-methoxy-benzyl)-9H-purine

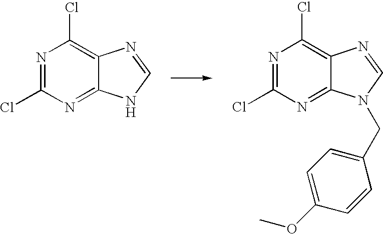

To DMSO (25 ml) was added K₂CO₃ (3.84 g, 27.8 mmol) followed by 2,6-dichloropurine (5 g, 26.5 mmol). The resulting suspension was stirred for 10 minutes, and then 4-methoxybenzyl chloride (3.59 ml, 26.5 mmol) was added and the reaction stirred at ambient temperature for 18 hours. Water (100 ml) was added to the reaction mixture and the product extracted with ethyl acetate (3×150 ml). The combined organic extracts were washed with saturated sodium chloride solution (2×100 ml), dried over MgSO₄ and concentrated in vacuo. The residue was purified via flash chromatography using 40% ethyl acetate/hexane as eluent. 3.72 g, 46% yield.

LC-MS retention time 2.366 min (270 nm) [M+H]⁺ 311, 309 (run time 3.75 min)

### Step 2

### 6-(5-Benzyloxy-2,4-dichloro-phenyl)-2-chloro-9-(4-methoxy-benzyl)-9H-purine

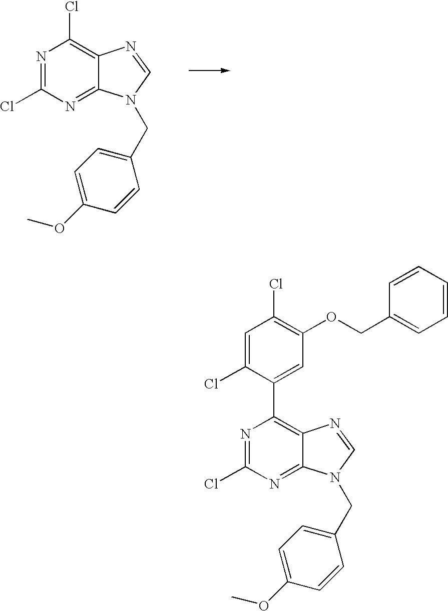

Potassium acetate (2.95 g, 30 mmol) was added to a solution of 1-Benzyloxy-2,4-dichloro-5-iodo-benzene (3.79 g, 10 mmol) (prepared as in example 2) and bis(pinacolato)diboron (2.67 g, 10.5 mmol) in DMF (50 ml) under a nitrogen atmosphere. Palladium acetate (112 mg, 5 mol %) was added and the mixture heated, oil bath temperature 90° C., for ˜18 hrs. The resulting solution solvents were removed in vacuo, and the residue taken up in ethyl acetate the solution was washed with water (3×) and saturated aqueous sodium chloride solution (1×). The solution was dried over anhydrous sodium sulphate and concentrated to a pale brown gum. The residue was taken up in 1,4-dioxan (50 ml) and 2,6-Dichloro-9-(4-methoxy-benzyl)-9H-purine (2.16 g, 7 mmol, 0.7 equiv) and aqueous potassium phosphate (21 mmol, 10.5 ml of a 2M solution) added, under a nitrogen atmosphere. The mixture was degassed and then dichloro bis(triphenylphosphine) palladium(II) (245 mg, 5 mol %) was added and the mixture heated, oil bath temperature 100° C., for 18 hrs. The mixture was allowed to cool and ethyl acetate (150 ml) added. The mixture was washed with saturated aqueous sodium chloride solution (5×100 ml), dried over MgSO₄ and concentrated in vacuo. The residue was purified via flash chromatography using 30% ethyl acetate/hexane as the eluent. 1.29 g, 35% yield.

LC retention time 2.849 min (270 nm) [M+H]⁺ 527, 525 (run time 3.75 min)

### Step 3

### 2,4-Dichloro-5-[2-chloro-9-(4-methoxy-benzyl)-9H-purin-6-yl]-phenol

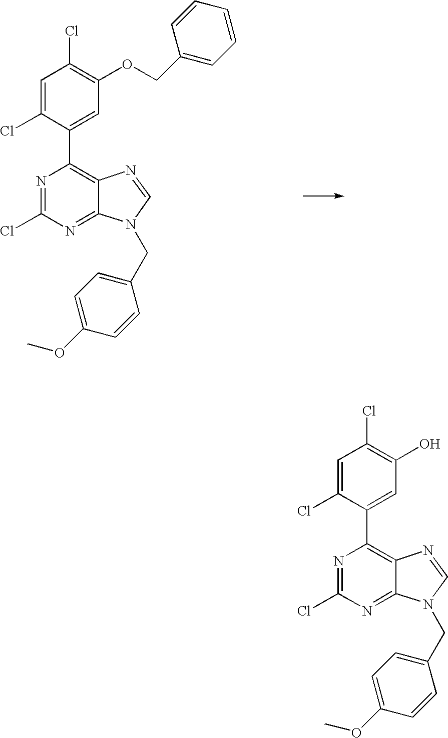

To dichloromethane (50 ml) was added 6-(5-Benzyloxy-2,4-dichloro-phenyl)-2-chloro-9-(4-methoxy-benzyl)-9H-purine (1.078 g, 2.05 mmol) and the mixture was cooled to −78° C. BCl₃ (10.25 mmol, 10.25 ml of a 1M solution in dichloromethane) was added drop wise and then cooling was removed allowing reaction to attain ambient temperature. The reaction was then cooled to −30° C., and quenched by the careful drop wise addition of water (50 ml). The dichloromethane layer was separated, washed with saturated sodium chloride solution (2×50 ml), dried using MgSO₄ and the solvent concentrated in vacuo. The residue was purified via flash chromatography using 50% ethyl acetate/hexane as the eluent. 0.554 g, 62% yield.

LC retention time 2.542 min (270 nm) [M+H]⁺ 437, 435 (run time 3.75 min)

### Step 4

### (3-{2,4-Dichloro-5-[2-chloro-9-(4-methoxy-benzyl)-9H-purin-6-yl]-phenoxy}-propyl)-dimethyl-amine

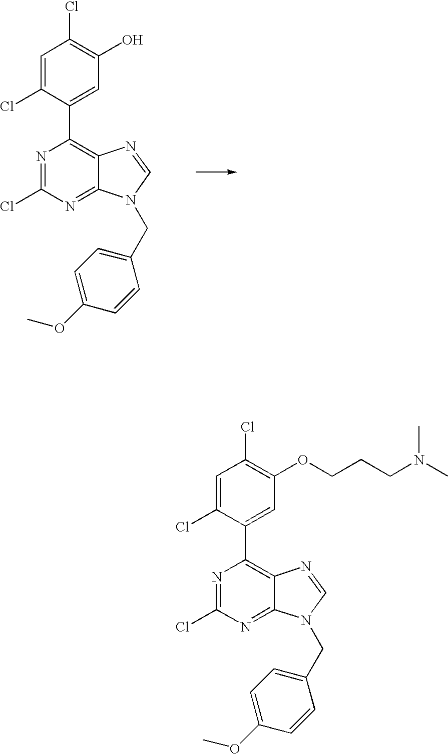

2,4-Dichloro-5-[2-chloro-9-(4-methoxy-benzyl)-9H-purin-6-yl]-phenol (175 mg, 0.402 mmol), 3-dimethylamino-1-propanol (50 mg, 0.482 mmol) and triphenylphosphine (158 mg, 0.603 mmol) were stirred in anhydrous tetrahydrofuran (2 ml) for 10 minutes and then the reaction mixture was cooled to 0° C. Diisopropyl azodicarboxylate (122 mg, 0.603 mmol) in anhydrous tetrahydrofuran (1 ml) was added drop wise, and after addition the cooling was removed and reaction attained ambient temperature. After 2 hours stirring at ambient temperature, the reaction mixture was partitioned between ethyl acetate and water. The organic layer was separated and washed with water, saturated sodium bicarbonate solution, saturated sodium chloride solution and dried over MgSO₄. The solvent was removed in vacuo and the residue was purified via flash chromatography using 5% methanol/dichloromethane as the eluent.

0.048 g 23% yield.

LC retention time 1.955 min (270 nm) [M+H]⁺ 522, 520 (run time 3.75 min)

### Step 5

### {3-[2,4-Dichloro-5-(2-chloro-9H-purin-6-yl)-phenoxy]-propyl}-dimethyl-amine

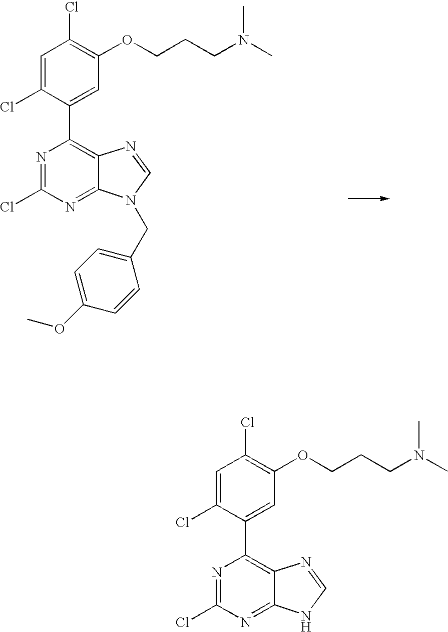

(3-{2,4-Dichloro-5-[2-chloro-9-(4-methoxy-benzyl)-9H-purin-6-yl]-phenoxy}-propyl)-dimethyl-amine (48 mg, 0.092 mmol) was heated in trifluoroacetic acid (5 ml) at 70° C. (oil bath temperature) for 3 hours. The reaction was cooled to ambient temperature and then carefully added to rapidly stirring ice/water (25 ml). This aqueous mixture was then carefully basified pH9 using ammonium hydroxide solution, and extracted using dichloromethane (2×20 ml). The combined extracts were dried using MgSO₄ and the solvent was removed in vacuo. The residue was purified by preparative HPLC at pH4.

LC retention time 1.621 min (270 nm) [M+H]⁺ 402, 400 (run time 3.75 min)

This compound has activity “A” in the fluorescence polarization assay described below.

### Example 35

### {2-[2,4-Dichloro-5-(2-chloro-9H-purin-6-yl)-phenoxy]-ethyl}-diethyl-amine

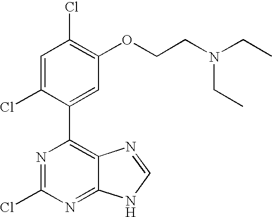

### Step 1

### (2-{2,4-Dichloro-5-[2-chloro-9-(4-methoxy-benzyl)-9H-purin-6-yl]-phenoxy}-ethyl)-diethyl-amine

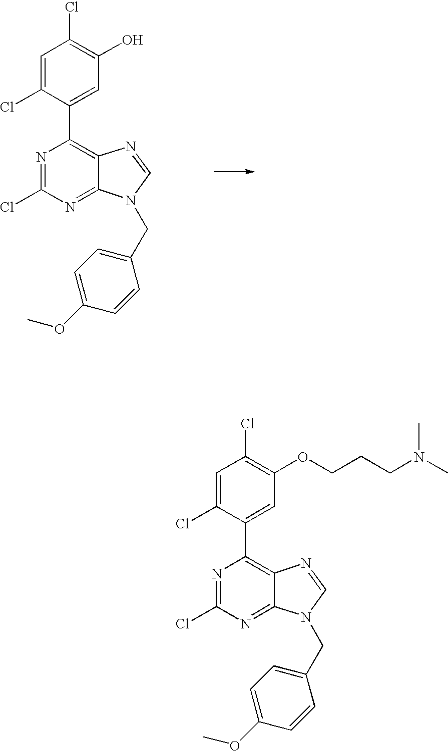

2,4-Dichloro-5-[2-chloro-9-(4-methoxy-benzyl)-9H-purin-6-yl]-phenol {prepared in Example 34, Step 3} (175 mg, 0.402 mmol), 2-(diethylamino)-ethanol (56 mg, 0.482 mmol) and triphenylphosphine (158 mg, 0.603 mmol) were stirred in anhydrous tetrahydrofuran (2 ml) for 10 minutes and then the reaction mixture was cooled to 0° C. Diisopropyl azodicarboxylate (122 mg, 0.603 mmol) in anhydrous tetrahydrofuran (1 ml) was added drop wise, and after addition the cooling was removed and reaction attained ambient temperature. After 2 hours stirring at ambient temperature, the reaction mixture was partitioned between ethyl acetate and water. The organic layer was separated and washed with water, saturated sodium bicarbonate solution, saturated sodium chloride solution and dried over MgSO₄. The solvent was removed in vacuo and the residue was purified on a MP-TsOH cartridge (2.5 g, ex Argonaut, Hengoed, UK).

0.200 g 93% yield.

LC retention time 1.963 min (270 nm) [M+H]⁺ 536, 534 (run time 3.75 min)

This material was used in the next step without further purification.

### Step 2

### (2-{2,4-Dichloro-5-[2-chloro-9-(4-methoxy-benzyl)-9H-purin-6-yl]-phenoxy}-ethyl)-diethyl-amine

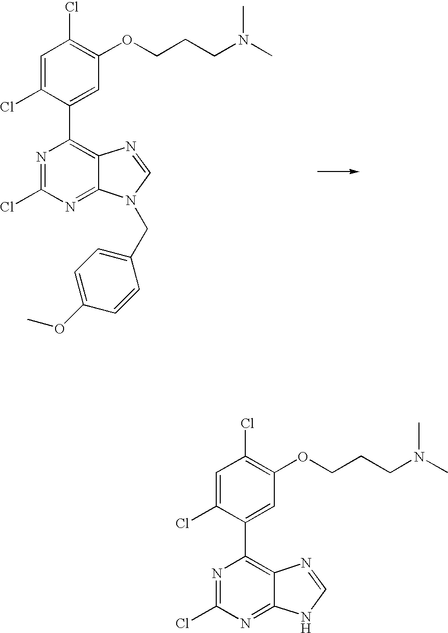

(2-{2,4-Dichloro-5-[2-chloro-9-(4-methoxy-benzyl)-9H-purin-6-yl]-phenoxy}-ethyl)-diethyl-amine (200 mg, 0.374 mmol) was heated in trifluoroacetic acid (7 ml) at 70° C. (oil bath temperature) for 18 hours. The reaction was cooled to ambient temperature and then carefully added to rapidly stirring ice/water (25 ml). This aqueous mixture was then carefully basified to pH9 using ammonium hydroxide solution, and extracted using dichloromethane (2×20 ml). The combined extracts were dried using MgSO₄ and the solvent was removed in vacuo. The residue was purified by preparative HPLC at pH4. The compound was further purified on a MP-TsOH cartridge (0.5 g ex Argonaut, Hengoed, UK).

LC retention time 1.642 min (270 nm) [M+H]⁺ 416, 414 (run time 3.75 min)

This compound has activity “A” in the fluorescence polarization assay described below.

### Example 36

### 2-Chloro-6-[2,4-dichloro-5-(2-pyrrolidin-1-yl-ethoxy)-phenyl]-9H-purine

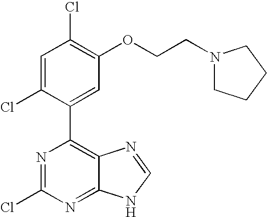

### Step 1

### 2-Chloro-6-[2,4-dichloro-5-(2-pyrrolidin-1-yl-ethoxy)-phenyl]-9-(4-methoxy-benzyl)-9H-purine

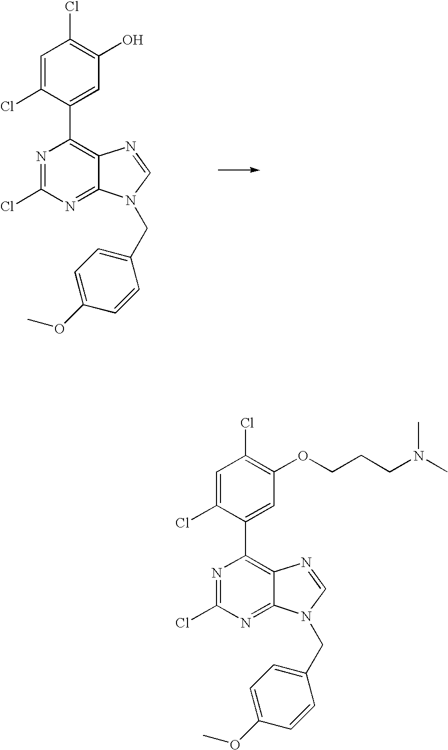

2,4-Dichloro-5-[2-chloro-9-(4-methoxy-benzyl)-9H-purin-6-yl]-phenol {prepared in Example 34, Step 3} (200 mg, 0.459 mmol), cesium carbonate (300 mg, 0.92 mmol) and 1-(2-chloroethyl)pyrrolidine hydrochloride (94 mg, 0.552 mmol) were heated at 140° C. (oil bath temperature) for 5 hours. The reaction mixture was cooled and partitioned between ethyl acetate and water. The organic layer was washed with saturated sodium chloride solution (5×50 ml), dried over MgSO₄ and concentrated in vacuo. The residue was purified on an ion exchange column (IST SCX II, 10 g ex Argonaut, Hengoed, UK). 0.135 g, 55% yield

LC retention time 1.967 min (270 nm) [M+H]⁺ 534, 532 (run time 3.75 min)

### Step 2

### 2-Chloro-6-[2,4-dichloro-5-(2-pyrrolidin-1-yl-ethoxy)-phenyl]-9H-purine

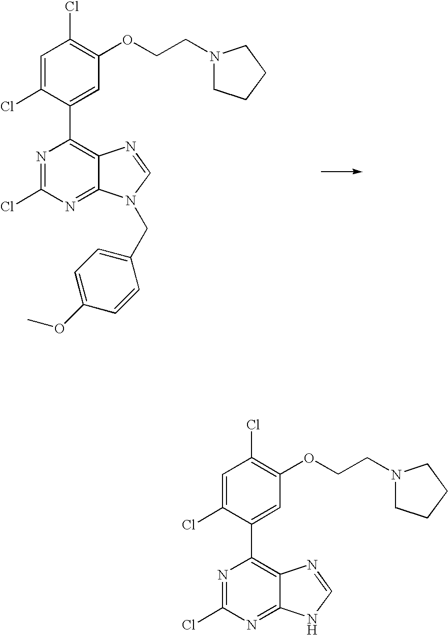

2-Chloro-6-[2,4-dichloro-5-(2-pyrrolidin-1-yl-ethoxy)-phenyl]-9-(4-methoxy-benzyl)-9H-purine (135 mg, 0.254 mmol) was heated in trifluoroacetic acid (7 ml) at 70° C. (oil bath temperature) for 18 hours. The reaction was cooled to ambient temperature and then carefully added to rapidly stirring ice/water (25 ml). This aqueous mixture was then carefully basified to pH9 using ammonium hydroxide solution, and extracted using dichloromethane (2×20 ml). The combined extracts were dried using MgSO₄ and the solvent was removed in vacuo. The residue was purified by preparative HPLC at pH4.

LC retention time 1.619 min (270 nm) [M+H]⁺ 414, 412 (run time 3.75 min)

This compound has activity “A” in the fluorescence polarization assay described below.

### Example 37

### 1-{2-[2,4-Dichloro-5-(2-chloro-9H-purin-6-yl)-phenoxy]-ethyl}-pyrrolidin-2-one

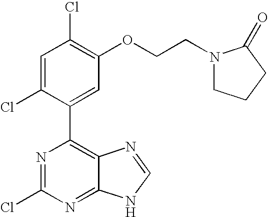

### Step 1

### 1-(2-Chloro-ethyl)-pyrrolidin-2-one

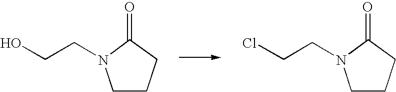

To a solution of 1-(2-hydroxyethyl)-2-pyrrolidinone (500 mg, 3.87 mmol) in chloroform (5 ml) was added thionyl chloride (0.491 ml, 4.26 mmol). The resultant solution was heated at reflux for 3 hours after which time no starting material remained. The reaction mixture was concentrated in vacuo and the residue purified by flash column chromatography using ethyl acetate as eluent to give the title compound as a pale brown liquid (530 mg, 93%).

### Step 2

### 1-(2-{2,4-Dichloro-5-[2-chloro-9-(4-methoxy-benzyl)-9H-purin-6-yl]-phenoxy}-ethyl)-pyrrolidin-2-one

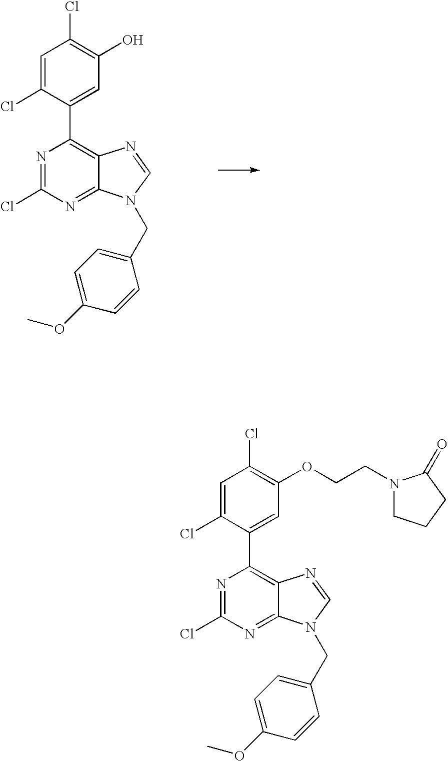

2,4-Dichloro-5-[2-chloro-9-(4-methoxy-benzyl)-9H-purin-6-yl]-phenol {prepared in Example 34, Step 3} (100 mg, 0.230 mmol), cesium carbonate (150 mg, 0.46 mmol) and 1-(2-chloroethyl)-pyrrolidin-2-one (37 mg, 0.253 mmol) were heated at 140° C. (oil bath temperature) for 3 hours. The reaction mixture was cooled and partitioned between ethyl acetate and water. The organic layer was washed with saturated sodium chloride solution (5×50 ml), dried over MgSO₄ and concentrated in vacuo. The residue was purified via flash chromatography eluting with 5% ethyl acetate/dichloromethane and then 5% methanol/dichloromethane. 0.120 g, 97% yield

LC retention time 2.551 min (270 nm) [M+H]⁺ 548, 546 (run time 3.75 min)

### Step 3

### 1-{2-[2,4-Dichloro-5-(2-chloro-9H-purin-6-yl)-phenoxy]-ethyl}-pyrrolidin-2-one

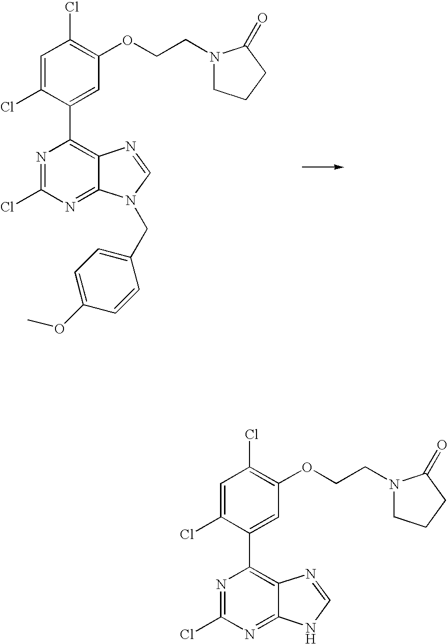

1-(2-{2,4-Dichloro-5-[2-chloro-9-(4-methoxy-benzyl)-9H-purin-6-yl]-phenoxy}-ethyl)-pyrrolidin-2-one (120 mg, 0.22 mmol) was heated in trifluoroacetic acid (5 ml) at 70° C. (oil bath temperature) for 3 hours. The reaction was cooled to ambient temperature and then carefully added to rapidly stirring ice/water (25 ml). This aqueous mixture was then carefully basified to pH9 using ammonium hydroxide solution, and extracted using dichloromethane (2×20 ml). The combined extracts were dried using MgSO₄ and the solvent was removed in vacuo. The residue was purified by preparative HPLC at pH4.

LC retention time 2.072 min (270 nm) [M+H]⁺ 428, 426 (run time 3.75 min)

This compound has activity “A” in the fluorescence polarization assay described below.

### Fluorescence Polarization Assay

Fluorescence polarization {also known as fluorescence anisotropy} measures the rotation of a fluorescing species in solution, where the larger molecule the more polarized the fluorescence emission. When the fluorophore is excited with polarized light, the emitted light is also polarized. The molecular size is proportional to the polarization of the fluorescence emission.

The fluoroscein-labelled probe-VER00051001-FAM-

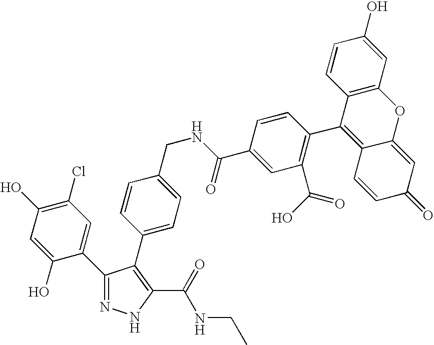

binds to HSP90 {full-length human, full-length yeast or N-terminal domain HSP90} and the anisotropy {rotation of the probe:protein complex} is measured.

Test compound is added to the assay plate, left to equilibrate and the anisotropy measured again. Any change in anisotropy is due to competitive binding of compound to HSP90, thereby releasing probe.

### Materials

Chemicals are of the highest purity commercially available and all aqueous solutions are made up in AR water.
1) Costar 96-well black assay plate #3915
2) Assay buffer of (a) 100 mM Tris pH7.4; (b) 20 mM KCl; (c) 6 mM MgCl₂. Stored at room temperature.
3) BSA (bovine serum albumen) 10 mg/ml (New England Biolabs # B9001S)
4) 20 mM probe in 100% DMSO stock concentration. Stored in the dark at RT. Working concentration is 200 nM diluted in AR water and stored at 4° C. Final concentration in assay 80 nM.
5) E. coli expressed human full-length HSP90 protein, purified >95% (see, e.g., Panaretou et al., 1998) and stored in 50 μL aliquots at −80° C.

### Protocol

1) Add 100 μl 1× buffer to wells 11A and 12A(═FPBLNK)
2) Prepare assay mix—all reagents are kept on ice with a lid on the bucket as the probe is light-sensitive.

i. Final Concⁿ

| 1x Hsp90 FP Buffer | 10 | ml | 1x | |
|---|---|---|---|---|
| BSA 10 mg/ml (NEB) | 5.0 | μl | 5 | μg/ml |
| Probe 200 μM | 4.0 | μl | 80 | nM |
| Human full-length Hsp90 | 6.25 | μl | 200 | nM |

3) Aliquot 100 μl assay mix to all other wells
4) Seal plate and leave in dark at room temp for 20 minutes to equilibrate
   Compound Dilution Plate—1×3 dilution series
1) In a clear 96-well v-bottom plate—{# VWR 007/008/257} add 10 μl 100% DMSO to wells B1 to H11
2) To wells A1 to A11 add 17.5 μl 100% DMSO
3) Add 2.5 μl cpd to A1. This gives 2.5 mM {50×} stock cpd—assuming cpds 20 mM.
4) Repeat for wells A2 to A10. Control in columns 11 and 12.
5) Transfer 5 μl from row A to row B- not column 12. Mix well.
6) Transfer 5 μl from row B to row C. Mix well.
7) Repeat to row G.
8) Do not add any compound to row H—this is the 0 row.
9) This produces a 1×3 dilution series from 50 μM to 0.07 μM.
10) In well B12 prepare 20 μl of 100 μM standard compound.
11) After first incubation the assay plate is read on a Fusion™ α-FP plate reader (Packard BioScience, Pangbourne, Berkshire, UK).
12) After the first read, 2 μl of diluted compound is added to each well for columns 1 to 10. In column 11 {provides standard curve} only add compound B11-H11. Add 2 μl of 100 mM standard cpd to wells B12-H12 {is positive control}
13) The Z′ factor is calculated from zero controls and positive wells. It typically gives a value of 0.7-0.9.

The compounds tested in the above assay were assigned to one of two activity ranges, namely A=<10 μM; B=>10 μM, and those assignments are reported above.

A growth inhibition assay was also employed for the evaluation of candidate HSP90 inhibitors:

### Assessment of Cytotoxicity by Sulforhodamine B (SRB) Assay: Calculation of 50% Inhibitory Concentration (IC₅₀).

### Day 1

1) Determine cell number by haemocytometer.
2) Using an 8 channel multipipettor, add 160 μl of the cell suspension (3600 cells/well or 2×10⁴ cells/ml) to each well of a 96-well microtitre plate.
3) Incubate overnight at 37° C. in a CO₂ incubator.

### Day 2

4) Stock solutions of drugs are prepared, and serial dilutions of each drug are performed in medium to give final concentrations in wells.
5) Using a multipipettor, 40 μl of drug (at 5× final concentration) is added to quadruplicate wells.
6) Control wells are at either side of the 96 well plates, where 40 μl of medium is added.
7) Incubate plates in CO₂ incubator for 4 days (48 hours).

### Day 6

8) Tip off medium into sink and immerse plate slowly into 10% ice cold trichloroacetic acid (TCA). Leave for about 30 mins on ice.
9) Wash plates three times in tap water by immersing the plates into baths of tap water and tipping it off.
10) Dry in incubator.
11) Add 100 μl of 0.4% SRB in 1% acetic acid to each well (except the last row (right hand) of the 96 well plate, this is the 0% control, ie no drug, no stain. The first row will be the 100% control with no drug, but with stain). Leave for 15 mins.
12) Wash off unbound SRB stain with four washes of 1% acetic acid.
13) Dry plates in incubator.
14) Solubilise SRB using 100 μl of 10 mM Tris base and put plates on plate shaker for 5 mins.
15) Determine absorbance at 540 nm using a plate reader. Calculate mean absorbance for quadruplicate wells and express as a percentage of value for control, untreated wells.
16) Plot % absorbance values versus log drug concentration and determine the IC₅₀.

### REFERENCES

A number of publications are cited above in order to more fully describe and disclose the invention and the state of the art to which the invention pertains. Full citations for these references are provided below. Each of these references is incorporated herein by reference in its entirety into the present disclosure.
Argon Y and Simen B B. 1999 “Grp94, an ER chaperone with protein and peptide binding properties”, Semin. Cell Dev. Biol., Vol. 10, pp. 495-505.
Bijlmakers M-JJE, Marsh M. 2000 “Hsp90 is essential for the synthesis and subsequent membrane association, but not the maintenance, of the Src-kinase p56lck”, Molecular Biology of the Cell, Vol. 11 (5), pp. 1585-1595.
Bucci M; Roviezzo F; Cicala C; Sessa W C, Cirino G. 2000 “Geldanamycin, an inhibitor of heat shock protein 90 (Hsp90) mediated signal transduction has anti-inflammatory effects and interacts with glucocorticoid receptor in vivo”, Brit. J. Pharmacol., Vol 131(1), pp. 13-16.
Chen C-F, Chen Y, Dai K D, Chen P-L, Riley D J and Lee W-H. 1996 “A new member of the hsp90 family of molecular chaperones interacts with the retinoblastoma protein during mitosis and after heat shock”, Mol. Cell. Biol., Vol. 16, pp. 4691-4699.
Chiosis G, Timaul M N, Lucas B, Munster P N, Zheng F F, Sepp-Lozenzino L and Rosen N. 2001 “A small molecule designed to bind to the adenine nucleotide pocket of HSP90 causes Her2 degradation and the growth arrest and differentiation of breast cancer cells”, Chem. Biol., Vol. 8, pp. 289-299.
Conroy S E and Latchman D S. 1996 “Do heat shock proteins have a role in breast cancer?”, Brit. J. Cancer, Vol. 74, pp. 717-721.
Felts S J, Owen B A L, Nguyen P, Trepel J, Donner D B and Toft D O. 2000 “The HSP90-related protein TRAP1 is a mitochondrial protein with distinct functional properties”, J. Biol. Chem., Vol. 5, pp. 3305-3312.
Fuller W, Cuthbert A W. 2000 “Post-translational disruption of the delta F508 cystic fibrosis transmembrane conductance regulator (CFTR)-molecular Chaperone complex with geldanamycin stabilizes delta F508 CFTR in the rabbit reticulocyte lysate”, J. Biol. Chem;. Vol 275(48), pp. 37462-37468.
Hickey E, Brandon S E, Smale G, Lloyd D and Weber L A. 1999 “Sequence and regulation of a gene encoding a human 89-kilodalton heat shock protein”, Mol. Cell. Biol., Vol. 9, pp. 2615-2626.
Hoang A T, Huang J. Rudra-Gonguly N. Zheng J. Powell W C, Rabindron S K, Wu C and Roy-Burman P. 2000 “A novel association between the human heat shock transcription factor I (HSF1) and prostate adenocarcinoma, Am. J. Pathol., Vol. 156, pp. 857-864.
Hostein I, Robertson D, Di Stefano F. Workman P and Clarke P A. 2001 “Inhibition of signal transduction by the HSP90 inhibitor 17-allylamino-17-demethoxygeldanamycin results in cytostasis and apoptosis”, Cancer Res., Vol. 61, pp. 4003-4009.
Hur E, Kim H-H, Choi S M, Kim J H, Yim S. Kwon H J, Choi Y. Kim D K, Lee M-O, Park H.2002 “Reduction of hypoxia-induced transcription through the repression of hypoxia-inducible factor-1α/aryl hydrocarbon receptor nuclear translocator DNA binding by the 90-kDa heat-shock protein inhibitor radicicol”, Mol. Pharmacol., Vol 62(5), pp. 975-982.
Hutter et al, 1996, Circulation, Vol. 94, pp. 1408.
Jameel A, Skilton R A, Campbell T A, Chander S K, Coombes R C and Luqmani Y A. 1992 “Clinical and biological significance of HSP89a in human breast cancer”, Int. J. Cancer, Vol. 50, pp. 409-415.
Jolly C and Morimoto R I. 2000 “Role of the heat shock response and molecular chaperones in oncogenesis and cell death”, J. Natl. Cancer Inst., Vol. 92, pp. 1564-1572.
Kawanishi K, Shiozaki H, Doki Y, Sakita I, Inoue M, Yano M, Tsujinata T, Shamma A and Monden M. 1999 “Prognostic significance of heat shock proteins 27 and 70 in patients with squamous cell carcinoma of the esophagus”, Cancer, Vol. 85, pp. 1649-1657.
Kelland L R, Abel G, McKeage M J, Jones M, Goddard P M, Valenti M, Murrer B A and Harrap K R. 1993 “Preclinical antitumour evaluation of bis-acetalo-amino-dichloro-cyclohexylamine platinum (IV): an orally active platinum drug”, Cancer Research, Vol. 53, pp. 2581-2586.
Kelland L R, Sharp S Y, Rogers P M, Myers T G and Workman P. 1999 “DT-diaphorase expression and tumor cell sensitivity to 17-allylamino, 17-demethoxygeldanamycin, an inhibitor of heat shock protein 90”, J. Natl. Cancer Inst., Vol. 91, pp. 1940-1949.
Kurebayashi J, Otsuki T, Kurosumi M, Soga S, Akinaga S, Sonoo, H. 2001 “A radicicol derivative, KF58333, inhibits expression of hypoxia-inducible factor-1α and vascular endothelial growth factor, angiogenesis and growth of human breast cancer xenografts”, Jap. J. Cancer Res., Vol 92(12), 1342-1351.
Kwon H J, Yoshida M, Abe K, Horinouchi S and Bepple T. 1992 “Radicicol, an agent inducing the reversal of transformed phentoype of src-transformed fibroblasts, Biosci., Biotechnol., Biochem., Vol. 56, pp. 538-539.
Lebeau J. Le Cholony C, Prosperi M T and Goubin G. 1991 “Constitutive overexpression of 89 kDa heat shock protein gene in the HBL100 mammary cell line converted to a tumorigenic phenotype by the EJ/T24 Harvey-ras oncogene”, Oncogene, Vol. 6, pp. 1125-1132.
Marcu M G, Chadli A, Bouhouche I, Catelli M and Neckers L. 2000a “The heat shock protein 90 antagonist novobiocin interacts with a previously unrecognized ATP-binding domain in the carboxyl terminus of the chaperone”, J. Biol. Chem., Vol. 275, pp. 37181-37186.
Marcu M G, Schulte T W and Neckers L. 2000b “Novobiocin and related coumarins and depletion of heat shock protein 90-dependent signaling proteins”, J. Natl. Cancer Inst., Vol. 92, pp. 242-248.
Martin K J, Kritzman B M, Price L M, Koh B. Kwan C P, Zhang X, MacKay A, O'Hare M J, Kaelin C M, Mutter G L, Pardee A B and Sager R. 2000 “Linking gene expression patterns to therapeutic groups in breast cancer”, Cancer Res., Vol. 60, pp. 2232-2238.
Neckers L, Schulte T W and Momnaaugh E. 1999 “Geldanamycin as a potential anti-cancer agent: its molecular target and biochemical activity”, Invest. New Drugs, Vol. 17, pp. 361-373.
Page J, Heath J, Fulton R, Yalkowsky E, Tabibi E, Tomaszewski J, Smith A and Rodman L. 1997 “Comparison of geldanamycin (NSC-122750) and 17-allylaminogeldanamycin (NSC-330507D) toxicity in rats”, Proc. Am. Assoc. Cancer Res., Vol. 38, pp. 308.
Panaretou B, Prodromou C, Roe S M, O'Brien R, Ladbury J E, Piper P W and Pearl L H. 1998 “ATP binding and hydrolysis are essential to the function of the HSP90 molecular chaperone in vivo”, EMBO J., Vol. 17, pp. 4829-4836.
Plumier et al, 1997, Cell. Stress Chap., Vol. 2, pp. 162
Pratt W B. 1997 “The role of the HSP90-based chaperone system in signal transduction by nuclear receptors and receptors signalling via MAP kinase”, Annu. Rev. Pharmacol. Toxicol., Vol. 37, pp. 297-326.
Prodromou C and Pearl LH. 2000a “Structure and in vivo function of HSP90”, Curr. Opin. Struct. Biol., Vol. 10, pp. 46-51.
Prodromou C, Roe S M, O'Brien R, Ladbury J E, Piper P W and Pearl L H. 1997 “Identification and structural characterization of the ATP/ADP-binding site in the HSP90 molecular chaperone”, Cell, Vol. 90, pp. 65-75.
Prodromou C, Panaretou B, Chohan S, Siligardi G, O'Brien R, Ladbury J E, Roe S M, Piper P W and Pearl L H. 2000b “The ATPase cycle of HSP90 drives a molecular ‘clamp’ via transient dimerization of the N-terminal domains”, EMBO J., Vol. 19, pp. 4383-4392.
Rajder et al, 2000, Ann. Neurol., Vol. 47, pp. 782.
Roe S M, Prodromou C, O'Brien R, Ladbury J E, Piper P W and Pearl L H. 1999 “Structural basis for inhibition of the HSP90 molecular chaperone by the antitumour antibiotics radicicol and geldanamycin”, J. Med. Chem., Vol. 42, pp. 260-266.
Rutherford S L and Lindquist S. 1998 “HSP90 as a capacitor for morphological evolution. Nature, Vol. 396, pp. 336-342.
Schulte T W, Akinaga S, Murakata T, Agatsuma T, Sugimoto S, Nakano H, Lee Y S, Simen B B, Argon Y, Felts S, Toft D O, Neckers L M and Sharma S V. 1999 “Interaction of radicicol with members of the heat shock protein 90 family of molecular chaperones”, Mol. Endocrinology, Vol. 13, pp. 1435-1448.
Schulte T W, Akinaga S, Soga S, Sullivan W, Sensgard B, Toft D and Neckers L M. 1998 “Antibiotic radicicol binds to the N-terminal domain of HSP90 and shares important biologic activities with geldanamcyin”, Cell Stress and ChaPerones, Vol. 3, pp. 100-108.
Schulte T W and Neckers L M. 1998 “The benzoquinone ansamycin 17-allylamino-17-deemthoxygeldanamcyin binds to HSP90 and shares important biologic activities with geldanamycin”, Cancer Chemother. Pharmacol., Vol. 42, pp. 273-279.
Sittler et al, 2001, Hum. Mol. Genet., Vol. 10, pp. 1307.
Smith D F. 2001 “Chaperones in signal transduction”, in: Molecular chaperones in the cell (P Lund, ed.; Oxford University Press, Oxford and NY), pp. 165-178.
Smith D F, Whitesell L and Katsanis E. 1998 “Molecular chaperones: Biology and prospects for pharmacological intervention”, Pharmacological Reviews, Vol. 50, pp. 493-513.
Song H Y, Dunbar J D, Zhang Y X, Guo D and Donner D B. 1995 “Identification of a protein with homology to hsp90 that binds the type 1 tumour necrosis factor receptor”, J. Biol. Chem., Vol. 270, pp. 3574-3581.
Stebbins C E, Russo A, Schneider C, Rosen N, Hartl F U and Pavletich N P. 1997 “Crystal structure of an HSP90-geldanamcyin complex: targeting of a protein chaperone by an antitumor agent”, Cell, Vol. 89, pp. 239-250.
Supko J G, Hickman R L, Grever M R and Malspeis L. 1995 “Preclinical pharmacologic evaluation of geldanamycin as an antitumour agent”, Cancer Chemother. Pharmacol., Vol. 36, pp. 305-315.
Tratzelt et al, 1995, Proc. Nat. Acad. Sci., Vol. 92, pp. 2944.
Trost et al, 1998, J. Clin. Invest., Vol. 101, pp. 855.
Tytell M and Hooper P L. 2001 “Heat shock proteins: new keys to the development of cytoprotective therapies”, Emerging Therapeutic Targets, Vol. 5, pp. 267-287.
Uehara U. Hori M, Takeuchi T and Umezawa H. 1986 “Phenotypic change from transformed to normal induced by benzoquinoid ansamycins accompanies inactivation of p60src in rat kidney cells infected with Rous sarcoma virus”, Mol. Cell. Biol., Vol. 6, pp. 2198-2206.
Waxman, Lloyd H. Inhibiting hepatitis C virus processing and replication. (Merck & Co., Inc., USA). PCT Int. Appl. (2002), WO 0207761
Winklhofer et al, 2001, J. Biol. Chem., Vol. 276, 45160.
Whitesell L, Mimnaugh E G, De Costa B. Myers C E and Neckers L M. 1994 “Inhibition of heat shock protein HSP90-pp60v-src heteroprotein complex formation by benzoquinone ansamycins: essential role for stress proteins in oncogenic transformation”, Proc. Natl. Acad. Sci. USA., Vol. 91, pp. 8324-8328.
Yorgin et al. 2000 “Effects of geldanamycin, a heat-shock protein 90-binding agent, on T cell function and T cell nonreceptor protein tyrosine kinases”, J. Immunol., Vol 164(6), pp. 2915-2923.
Young J C, Moarefi I and Hartl F U. 2001 “HSP90: a specialized but essential protein-folding tool”, J. Cell. Biol., Vol. 154, pp. 267-273.
Zhao J F, Nakano H and Sharma S. 1995 “Suppression of RAS and MOS transformation by radicicol”, Oncogene, Vol. 11, pp. 161-173.

1. A compound of formula (I), or a salt, N-oxide, hydrate, or solvate thereof: 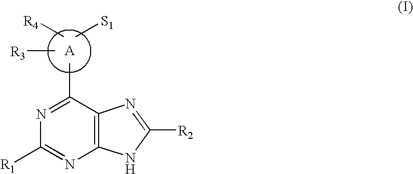
wherein
ring A is an aryl or heteroaryl ring or ring system;
R₁ is hydrogen, fluoro, chloro, bromo, or a radical of formula (1A):
—X-Alk¹-(Z)ₘ-(Alk²)ₙ-Q (IA)
wherein
X is a bond, —O—, —S— —S(O)—, —SO₂—, or —NH—,
Z is —O—, —S—, —(C═O)—, —(C═S)—, —S(O)—, —SO₂—, —NR^{A}—, or, in either orientation —C(═O)O—, —C(═O)NR^{A}—, —C(═S)NR^{A}—, —SO₂NR^{A}—, —NR^{A}C(═O)—, or —NR^{A}SO₂— wherein R^{A} is hydrogen or C₁-C₆ alkyl in which one or more hydrogens is optionally substituted by fluorine;
Alk¹ and Alk² are optionally substituted divalent C₁-C₃ alkylene or C₂-C₃ alkenylene radicals,
m and n are independently 0 or 1, and
Q is hydrogen or an optionally substituted carbocyclic or heterocyclic radical;
R₂ is cyano (—CN), fluoro, chloro, bromo, methyl, ethyl, —OH, —CH₂OH, —C(═O)NH₂, —C(═O)H, —C(═O)CH₃, or —NH₂;
R₃ and R₄ are independently selected from hydrogen, fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or R₃ and R₄ together represent a carbocyclic or heterocyclic ring fused to ring A, or methylenedioxy (—OCH₂O—) or ethylenedioxy (—OCH₂CH₂O—) in either of which one or more hydrogens are optionally replaced by fluorine;
S₁ is hydrogen, or a substituent selected from fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or S₁ and R₃, or S₁ and R₄, together represent methylenedioxy (—OCH₂O—) or ethylenedioxy ((—OCH₂ CH₂O—) in either of which one or more hydrogens are optionally replaced by fluorine; or S₁ is a radical of formula (IB):
-(Alk³)ₚ-(Z¹)_{q}-(Alk⁴)ᵣ-Q¹ (IB)
wherein
p, q and r are independently 0 or 1;
(a) when p is 0 or 1, and q is 1, and r is 0 or 1:
Z¹ is selected from the group of divalent radicals consisting of (i) —S—, —(C═O)—, —(C═S)—, —S(O)— and —SO₂— and (ii) —N(R^{A})C(═O)—* wherein the bond marked * is attached to Q¹ and (iii) in either orientation, —C(═O)O—, —C(═S)NR_{A}—, and —SO₂NR^{A}—; and Q¹ is (i) hydrogen or an optional substituent; or (ii) an optionally substituted carbocyclic or heterocyclic radical; or (iii) a radical —CH₂[O(CH₂)_{w}]ₓZ² wherein Z² is H, —OH or O(C₁-C₃alkyl) wherein x and w are independently 1, 2 or 3; or
(b) when p is 1, and q is 1, and r is 0 or 1:
Z is —O—, and Q¹ is (i) hydrogen or an optional substituent which is not linked to -(Alk³)ₚ-(Z¹)_{q}-(Alk⁴)ᵣ— through a nitrogen atom; or (ii) an optionally substituted carbocyclic radical; or (iii) an optionally substituted heterocyclic ring of 5 or 6 ring atoms which is not linked to -(Alk³)ₚ-(Z¹)_{q}-(Alk⁴)ᵣ— through a ring nitrogen; or (iv) a radical —CH₂[O(CH₂)_{w}]ₓZ² wherein Z² is H, —OH or O(C₁-C₃alkyl) wherein x and w are independently 1, 2 or 3. or
(c) when p is 1, and q is 1, and r is 0 or 1:
Z¹ is —NR^{A}— or —C(═O)N(R^{A})—* wherein the bond marked * is attached to Q¹ and Q¹ is a radical CH₂[O(CH₂)_{w}]ₓZ² wherein Z² is H, —OH or —O(C₁-C₃alkyl) wherein x and
w are independently 1, 2 or 3. or
(d) when p is 0, and q is 1, and r is 0 or 1:
Z¹ is —O— or —NR^{A}— and Q¹ is (i) hydrogen or an optional substituent which is not linked to -(Alk³)ₚ-(Z¹)_{q}-(Alk⁴)ᵣ— through a nitrogen atom; or (ii) Q¹ and R^{A}, taken together with the nitrogen to which they are attached form an optionally substituted heterocyclic ring of 5 or 6 ring atoms; or (iii) a radical —CH₂[O(CH₂)_{w}]ₓZ² wherein Z² is H, —OH or O(C₁-C₃alkyl) wherein x and w are independently 1, 2 or 3; or
(e) when p is 0 or 1, q is 0, and r is 0 or 1:
Q¹ is (i) hydrogen or an optional substituent which is not linked to -(Alk³)ₚ-(Z¹)_{q}-(Alk⁴)ᵣ— through a nitrogen atom or (ii) an optionally substituted carbocyclic radical; or (iii) an optionally substituted heterocyclic of 5 or 6 ring atoms which is not linked to -(Alk³)ₚ-(Z¹)_{q}-(Alk⁴)ᵣ— through a ring nitrogen; or (iv) a radical —CH₂[O(CH₂)_{w}]ₓZ² wherein Z² is H, —OH or O(C₁-C₃alkyl) wherein x and w are independently 1, 2 or 3;
R^{A} is hydrogen or C₁-C₃ alkyl optionally substituted with one or more fluorine substituents; and
Alk³ and Alk⁴ are divalent C₁-C₃ alkylene or C₂-C₃ alkenylene radicals, each optionally substituted by one or two substituents selected from fluoro, chloro, C₁-C₃alkyl optionally substituted with one or more fluorine substituents, C₁-C₃alkoxy optionally substituted with one or more fluorine substituents.

2. A compound as claimed in claim 1 wherein R₂ is hydrogen.

3. A compound as claimed in claim 2 wherein, in the group R₁: X is a bond, p is 1, and Z¹ is —O—, —S—, —(C═O)—, —(C═S)—, —SO₂—, —C(═O)O—, —C(═O)NR^{A}—, —C(═S)NR^{A}—, —SO₂NR^{A}—, —NR^{A}C(═O)—, —NR^{A}SO₂— or —NR^{A}— wherein R^{A} is hydrogen or C₁-C₆ alkyl.

4. A compound as claimed in claim 3 wherein, in the group R₂, S₁ is a radical of formula (IB) wherein: p is 0 or 1, and q is 1, and r is 0 or 1, Z¹ is selected from the group of divalent radicals consisting of (i) —S—, —(C═O)—, —(C═S)—, and —SO₂— and (ii) —N(R^{A})C(═O)—* wherein the bond marked * is attached to Q¹ and (iii) in either orientation, —C(═O)O—, —C(═S)NR^{A}— and —SO₂NR^{A}—; and Q¹ is (i) hydrogen or (ii) an optionally substituted carbocyclic or heterocyclic radical.

5. A compound as claimed in claim 3 wherein, in the group R₂, S₁ is a radical of formula (IB) wherein p is 1, and q is 1, and r is 0 or 1, Z¹ is —O—, and Q¹ is (i) hydrogen or (ii) an optionally substituted carbocyclic radical; or (iii) an optionally substituted heterocyclic ring of 5 or 6 ring atoms which is not linked to -(Alk³)ₚ-(Z¹)_{q}-(Alk⁴)ᵣ— through a ring nitrogen.

6. A compound as claimed in claim 3 wherein, in the group R₂, S₁ is a radical of formula (IB) wherein p is 0 or 1, q is 0, and r is 0 or 1, and Q¹ is (i) hydrogen or (ii) an optionally substituted carbocyclic radical; or (iii) an optionally substituted heterocyclic of 5 or 6 ring atoms which is not linked to -(Alk³)ₚ-(Z¹)_{q}-(Alk⁴)ᵣ— through a ring nitrogen.

7. A compound as claimed in claim 1 wherein ring A is a phenyl ring.

8. A compound as claimed in claim 1 wherein the radical comprising ring A and substituents R₃, R₄ and S₁ is a radical of formula (IC), 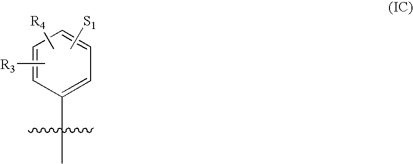
wherein
R₃ and R₄ are as defined in claim 1, and
S₁ is hydrogen, or a substituent selected from fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or S₁ and R₃, or S₁ and R₄, together represent methylenedioxy (—OCH₂O—) or ethylenedioxy ((—OCH₂ CH₂O—) in either of which one or more hydrogens are optionally replaced by fluorine; or S₁ is a radical of formula (IB):
-(Alk³)ₚ-(Z¹)_{q}-(Alk⁴)ᵣ-Q¹ (IB)
wherein
p, q and r are independently 0 or 1;
Z¹ is —O—, —S—, —(C═O)—, —(C═S)—, —S(O)—, —SO₂—, —NR^{A}—, or, in either orientation, —C(═O)N(R^{A})— or —SO₂NR^{A}—;
Q¹ is (i) hydrogen or an optional substituent; or (ii) an optionally substituted carbocyclic or heterocyclic radical; or (iii) a radical —CH₂[O(CH₂)_{w}]ₓZ² wherein Z² is H, —OH or O(C₁-C₃alkyl) wherein x and w are independently 1, 2 or 3;
R^{A} is hydrogen or C₁-C₃ alkyl optionally substituted with one or more fluorine substituents; and
Alk³ and Alk⁴ are divalent C₁-C₃ alkylene or C₂-C₃ alkenylene radicals, each optionally substituted by one or two substituents selected from fluoro, chloro, C₁-C₃alkyl optionally substituted with one or more fluorine substituents, C₁-C₃alkoxy optionally substituted with one or more fluorine substituents.

9. A compound as claimed in claim 7 wherein R₃ is in the ortho position and R₄ in the para position.

10. A compound as claimed in claim 7 wherein S₁ is in the meta position of the phenyl ring.

11. A compound as claimed in claim 1 wherein R₃ and/or R₄ is/are selected from fluoro, chloro, bromo and methyl.

12. A compound as claimed in claim 1 wherein R₁ is a radical of formula —W-Alk⁵-B wherein W is —O— or —S—, Alk5 is a straight or branched divalent C₁-C₆ alkylene radical in which one or more hydrogen atoms is/are replaced by fluorine atoms, and B hydrogen, —NH₂, —NHR^{A}, NHR^{A}R^{B} wherein R^{A} and R^{B} are independently hydrogen or C₁-C₆ alkyl or C₁-C₆ alkyl in which one or more hydrogen atoms is/are replaced by fluorine atoms, or R^{A} and R^{B} together with the nitrogen to which they are attached form a saturated 5- or 6-membered heterocyclic ring.

13. A compound as claimed in claim 1 wherein R₁ is methoxy, ethoxy, methylthio or ethylthio,

14. A compound as claimed in claim 1 which is the subject of any of the Examples herein.

15. A pharmaceutical or veterinary composition comprising a compound as claimed in claim 1, together with one or more pharmaceutically or veterinarily acceptable carriers and/or excipients.

16. (canceled)

17. A method of treatment of diseases which are responsive to inhibition of HSP90 activity in mammals, which method comprises administering to the mammal an amount of a compound as claimed in claim 1 effective to inhibit said HSP90 activity.

18. The method as claimed claim 17 for immunosuppression or the treatment of viral disease, or for co-therapy with antifungal agents in the treatment of fungal infection, inflammatory diseases such as rheumatoid arthritis, asthma, multiple sclerosis, Type I diabetes, lupus, psoriasis and inflammatory bowel disease; cystic fibrosis angiogenesis-related disease such as diabetic retinopathy, haemangiomas, and endometriosis; or for protection of normal cells against chemotherapy-induced toxicity; or diseases where failure to undergo apoptosis is an underlying factor; or protection from hypoxia-ischemic injury due to elevation of Hsp70 in the heart and brain; scrapie/CJD, Huntingdon's or Alzheimer's disease.

19. The method as claimed claim 17, for the treatment of cancer.
